(19) United States
(12) Patent Application Publication
(10) Pub. No.: US 2020/0008916 A1
(43) Pub. Date: Jan. 9, 2020

(54) METHODS FOR IMPROVED REPRODUCTIVE MANAGEMENT OF RUMINANT UNGULATES

(71) Applicant: The Penn State Research Foundation, University Park, PA (US)

(72) Inventors: Troy L. Ott, Boalsburg, PA (US); Roy H. Hammerstedt, Boalsburg, PA (US)

(21) Appl. No.: 16/470,407

(22) PCT Filed: Dec. 18, 2017
(86) PCT No.: PCT/US2017/067054

§ 371 Date: Jun. 17, 2019

Related U.S. Application Data
(60) Provisional application No. 62/435,432, filed on Dec. 16, 2016.

(51) Int. Cl. A61D 17/00; G01N 33/68; G01N 33/50; G06K 19/06; G06K 19/07; G16H 40/67

(52) U.S. Cl. CPC A61D 17/002; G01N 33/689; G01N 33/5091; A61D 19/02; G06K 19/0723; G16H 40/67; G06K 19/06037

(57) ABSTRACT

Methods according to aspects of the present invention relate to the determination of whether a female ruminant ungulate is open or not open using flow cytometry to detect an expression level of an interferon-stimulated gene in leukocytes of a biological sample from the female ruminant ungulate.

### REFERENCE TO RELATED APPLICATION

This application claims priority from U.S. Provisional Patent Application Ser. No. 62/435,432, filed Dec. 16, 2016, the entire content of which is incorporated herein by reference.

### GOVERNMENT SUPPORT

This invention was made with government support under Hatch Act Project No. PEN04511 awarded by the United States Department of Agriculture. The Government has certain rights in the invention.

### BACKGROUND OF THE INVENTION

Early determination of pregnancy status is critical for profitable animal production. Pregnancies in cattle have historically been determined by palpation of uterine contents per rectum, but palpation methods cannot determine whether conception occurred until about 30 days after insemination. Palpation is not practicable for other female ruminant ungulates (e.g., sheep, goats, deer, elk and bison). In these species, as well as cattle, detection of the placental protein pregnancy specific protein B (PSPB) by ELISA allows for the detection of pregnancy as early as 28-30 days after conception. Such ELISAs allow for pregnancy testing. With either palpation or a PSPB ELISA test, dairy cows can be artificially inseminated unsuccessfully about 3-4 times before a farmer typically makes the decision to stop breeding the cow and remove her from the herd (culling) for infertility. Improved methods of reproductive management could increase the number of artificial insemination attempts for an individual dairy cow (or other female ruminant ungulate) prior to reaching the culling decision (typically between 180-200 days after calving for dairy cows). Similarly, for beef cattle, producers have 2-3 insemination attempts to achieve a yearly calving interval before they reach a decision to cull the cow for infertility. Thus, for cattle, as well as for other commercially significant ruminant ungulate populations, reproductive management would be greatly improved and reproductive culls reduced if farmers could increase the number of inseminations prior to the point where they are compelled to cull the animal from the herd for infertility. There is a continuing need for methods for improved reproductive management of female ruminant ungulates.

### SUMMARY OF THE INVENTION

Various aspects of the present invention relate to methods of determining whether a female ruminant ungulate is “open” or “not open” following implementation of a method to establish pregnancy, such as insemination or embryo transfer. Pregnancy tests are administered at physiologically relevant stages of pregnancy (e.g., typically >28 days after insemination in cattle). The various inventive methods described herein relate to “open” tests, which occur at different physiologically relevant time points (e.g., typically <25 days after insemination in cattle).

The term “open” is used herein to describe a female ruminant ungulate subject that has undergone implementation of a method to establish pregnancy, where the subject is not far enough along in its pregnancy for a pregnancy test (e.g., by palpation or ELISA) to provide a reliable result. The term “open” as used herein refers to a female ruminant ungulate (i.e., subject) that underwent implementation of a method to establish pregnancy but is not pregnant and that, following determination of “open” status, can undergo another implementation of a method to establish pregnancy. Open subjects include subjects that were inseminated and did not conceive and subjects that were inseminated and conceived but that do not carry an embryo. Open subjects also include subjects that underwent embryo transfer and that lost the embryo. An open cow, for example, can be a cow that was inseminated but that does not carry an embryo 18-20 days after insemination. The term “not open” as used herein refers both to a female ruminant ungulate (i.e., subject) that is pregnant with a viable or non-viable pregnancy and to a female ruminant ungulate that either conceived or underwent embryo transfer but that recently lost its embryo, e.g., within about 24-48 hours of drawing a sample from the subject. A female ruminant ungulate that is not open should not be artificially inseminated or undergo additional embryo transfer because doing so is unlikely to result in conception and may terminate an existing pregnancy.

The present invention describes methods for identification of open subjects. Methods are provided according to aspects of the present invention which include drawing less than about 200 μL of blood from a female ruminant ungulate such as about 100 μL, less than about 100 μL, or about 10 μL to about 120 μL. Drawing small amounts of blood can decrease assay costs and facilitate sample processing. Methods are provided according to aspects of the present invention which include drawing blood from a female ruminant ungulate and then immediately contacting the blood with a fixative (e.g., within about 5 minutes such as within about 2 minutes, about 1 minute, about 45 seconds, or even within about 30 seconds of drawing the blood). Immediately contacting the blood with a fixative can preserve labile analytes in the blood for further analysis. For example, according to aspects of the present invention, interferon-induced analytes are identified in relatively small volumes of blood. Immediately contacting the blood with a fixative can also stabilize interferon-induced analytes therein, which could otherwise become degraded, denatured, and/or aggregated, thereby impairing the ability to measure the interferon-induced analytes. Methods are provided according to aspects of the present invention which include inserting a sample (e.g., blood sample) into a sample container indelibly labeled with a unique identifier (e.g., barcode), which is associated with the subject number, thereby allowing automated tracking of the sample from the point of collection to sample analysis (e.g., from farm to lab). Methods are provided according to aspects of the present invention which include processing a sample in a single sample container, e.g., beginning with fixing the sample through analysis of the sample by flow cytometry, through an automated learning algorithm that will calculate baseline and call sample reads. Maintaining a sample from the point of collection through processing and analysis in a single sample container will reduce costs, reduce sample loss, and reduce the introduction of error, which commonly results from hand-labeled samples and multiple sample transfers between labeled tubes. Furthermore, reduction in sample volume collected greatly reduces cost of analysis and provides a welfare benefit by reducing blood volume drawn by up to 100-fold or more. Methods are provided according to aspects of the present invention which include the flow cytometric analysis of a sample. Rapid sample fixation coupled with flow cytometry allows for the detection of interferon-induced analytes within individual cells. This provides a number of important benefits. For example, analytes are fixed in situ thereby reducing losses that invariably occur with cell lysates and/or extracts in solution-based assays. Furthermore, in situ fixation decreases the probability that the interferon-induced analytes will degrade, aggregate, or unfold prior to their detection. Flow cytometric analysis of individual cells from an individual sample provides logarithmic increases in the ‘technical replication’ of an individual analysis. Flow cytometry allows for the detection of “outlier” populations, e.g., populations of cells in non-parametric distributions that are otherwise masked when analyzing mean concentrations such as in commonly-used solution-based PCR and ELISA assays. Methods are provided according to aspects of the present invention which include the determination of whether a sample is from an open or not open subject following a method to establish a pregnancy based on either Bayesian analysis or the analysis of flow cytometry data obtained for the sample against parameters defined by machine learning software. The use of Bayesian analysis and machine learning software increases the accuracy of determinations relative to binary comparisons commonly used in PCR- and ELISA-based analyses. Methods are provided according to aspects of the present invention which include cloud-based sample logging, data analysis, and storage from farm to testing site and back to farm, thereby allowing sample tracking to document process integrity and/or to provide a single repository of flow cytometry data and parameters relevant to sample analysis, which allows for the iterative improvement of flow-cytometry-based determinations.

Methods of improved reproductive management of a female ruminant ungulate are provided according to aspects of the present invention which include contacting a biological sample with a binding agent specific for an interferon-induced analyte to be assayed in the biological sample, thereby producing a complex between the binding agent and the interferon-induced analyte, wherein the biological sample includes leukocytes of a female subject, and the subject is a female ruminant ungulate that underwent implementation of a method to establish pregnancy; and detecting the complex using flow cytometry, thereby allowing for a determination of whether the subject is open or not open.

Methods of improved reproductive management of a female ruminant ungulate are provided according to aspects of the present invention which include communicating to the owner of the subject or an agent thereof a determination that the subject is open or not open.

Methods of improved reproductive management of a female ruminant ungulate are provided according to aspects of the present invention wherein the subject is a cow. Methods of improved reproductive management of a female ruminant ungulate are provided according to aspects of the present invention wherein the leukocytes are characterized by the fact that they were drawn from the cow 17 to 21 days after insemination of the cow or 10 to 16 days after embryo transfer into the cow. Methods of improved reproductive management of a female ruminant ungulate are provided according to aspects of the present invention wherein the leukocytes are characterized by the fact that they were drawn from the cow 18, 19, or 20 days after insemination of the cow or 11, 12, or 13 days after embryo transfer into the cow.

Methods of improved reproductive management of a female ruminant ungulate are provided according to aspects of the present invention wherein the subject is a sheep. Methods of improved reproductive management of a female ruminant ungulate are provided according to aspects of the present invention wherein the leukocytes are characterized by the fact that they were drawn from the sheep 13 to 20 days after insemination of the sheep or 6 to 15 days after embryo transfer into the sheep. Methods of improved reproductive management of a female ruminant ungulate are provided according to aspects of the present invention wherein the leukocytes are characterized by the fact that they were drawn from the sheep 14, 15, or 16 days after insemination of the sheep or 7, 8, or 9 days after embryo transfer into the sheep.

Methods of improved reproductive management of a female ruminant ungulate are provided according to aspects of the present invention wherein: it is determined that the female ruminant ungulate is open; the method includes contacting a second biological sample with the binding agent specific for the interferon-induced analyte to be assayed in the biological sample, thereby producing a second complex between the binding agent and the interferon-induced analyte; the method includes detecting the second complex using flow cytometry, thereby allowing for a determination of whether the subject is open or not open; the second biological sample includes leukocytes of the subject; the leukocytes of the second biological sample are characterized by the fact that they were drawn from the subject after the subject had undergone a second implementation of a method to establish pregnancy; the second implementation of a method to establish pregnancy occurred after detecting the complex using flow cytometry; and the second implementation of a method to establish pregnancy occurred during the estrus cycle immediately following the estrus cycle during which the first implementation of a method to establish pregnancy occurred.

Methods of improved reproductive management of a female ruminant ungulate are provided according to aspects of the present invention wherein the first method to establish pregnancy and the second method to establish pregnancy are both inseminations; the second insemination occurred 21-24 days after the first insemination occurred; the leukocytes of the biological sample are characterized by the fact that they were drawn from the subject during a first estrus of the subject; the first insemination occurred during the first estrus of the subject; the second insemination occurred during a second estrus of the subject; and the first estrus and the second estrus are estruses of consecutive estrus cycles.

Methods of improved reproductive management of a female ruminant ungulate are provided according to aspects of the present invention wherein the method to establish pregnancy and the second method to establish pregnancy are both embryo transfers; the second embryo transfer occurred 21-24 days after the first embryo transfer occurred; the leukocytes of the biological sample are characterized by the fact that they were drawn from the subject during a first estrus of the subject; the first embryo transfer occurred during the first estrus of the subject; the second embryo transfer occurred during a second estrus of the subject; and the first estrus and the second estrus are estruses of consecutive estrus cycles.

Methods of improved reproductive management of a female ruminant ungulate are provided according to aspects of the present invention wherein the binding agent is specific for an interferon-induced analyte selected from the group consisting of interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR) or mRNA corresponding to an interferon-induced analyte selected from the group consisting of interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR).

Methods of improved reproductive management of a female ruminant ungulate are provided according to aspects of the present invention wherein the analyte is a protein.

Methods of improved reproductive management of a female ruminant ungulate are provided according to aspects of the present invention wherein the binding agent includes a monoclonal antibody, an antigen-binding portion of a monoclonal antibody, or a polyclonal antibody. Methods of improved reproductive management of a female ruminant ungulate are provided according to aspects of the present invention wherein the binding agent is an anti-ISG15 antibody, an anti-Mx1 antibody, an anti-Mx2 antibody, or a fluorescently-labeled analogue of any one of the foregoing.

Methods of improved reproductive management of a female ruminant ungulate are provided according to aspects of the present invention wherein the leukocytes of the biological sample are characterized by the fact that they are from about 0.1-0.2 mL blood that was drawn from the subject. Methods of improved reproductive management of a female ruminant ungulate are provided according to aspects of the present invention wherein the leukocytes are peripheral blood leukocytes. Methods of improved reproductive management of a female ruminant ungulate are provided according to aspects of the present invention wherein the leukocytes are characterized by the fact that they were obtained from milk or secretions from the reproductive tract.

Methods of improved reproductive management of a female ruminant ungulate are provided according to aspects of the present invention which include fixing and/or permeabilizing the leukocytes.

Methods of improved reproductive management of a female ruminant ungulate are provided according to aspects of the present invention which include contacting the leukocytes with a plurality of probes, wherein the plurality of probes includes a probe that specifically labels double stranded nucleic acids, thereby allowing the identification of isolated, mononuclear cells by flow cytometry.

Methods of improved reproductive management of a female ruminant ungulate are provided according to aspects of the present invention which include contacting the leukocytes with a plurality of probes, wherein the plurality of probes includes a probe that specifically labels a constitutively-expressed intracellular protein, thereby allowing the identification of permeabilized cells by flow cytometry.

Methods of improved reproductive management of a female ruminant ungulate are provided according to aspects of the present invention which include contacting the leukocytes with a plurality of probes, wherein the plurality of probes includes a probe that specifically labels a protein expressed by lymphocytes, monocytes, eosinophils, or granulocytes, thereby allowing the identification of lymphocytes, monocytes, eosinophils, or granulocytes by flow cytometry.

Methods of improved reproductive management of a female ruminant ungulate are provided according to aspects of the present invention which include contacting the leukocytes with a blocking reagent selected from the group consisting of milk, casein, serum, and a purified protein fraction of milk, casein, or serum.

Methods of analyzing leukocytes of a female subject are provided according to aspects of the present invention which include providing a biological sample including leukocytes of the subject, wherein the leukocytes are characterized by the fact that they were drawn from the subject after the subject underwent a implementation of a method to establish a pregnancy, and the subject is a female ruminant ungulate; fixing and permeabilizing the leukocytes; contacting the leukocytes with a binding agent, wherein the binding agent specifically labels a protein or mRNA corresponding to interferon stimulated gene 15 (ISG15), Mx1 protein (Mx1), Mx2 protein (Mx2), receptor transporter protein-4 (RTP4), 2′,5′ oligoadenylate synthetase (OAS), or protein kinase R (PKR), thereby allowing the identification of leukocytes that express one of the foregoing gene products by flow cytometry; analyzing the fluorescently-labeled sample by flow cytometry, thereby obtaining new flow cytometry data; and performing a second implementation of a method to establish a pregnancy in the subject after analyzing the fluorescently-labeled sample if the new flow cytometry data indicates that the subject is open.

Methods of analyzing leukocytes of a female subject are provided according to aspects of the present invention wherein the method to establish a pregnancy was a first insemination; and the second insemination of the subject takes place 21-24 days after the first insemination.

Methods of analyzing leukocytes of a female subject are provided according to aspects of the present invention wherein the method to establish a pregnancy was a first embryo transfer; and a second embryo transfer into the subject takes place 20-30 days after the first embryo transfer.

Methods are provided according to aspects of the present invention wherein the subject is not administered gonadotropin releasing hormone between the first implementation of a method to establish a pregnancy and the second implementation of a method to establish a pregnancy.

Methods are provided according to aspects of the present invention wherein the subject is administered prostaglandin F2α between the first implementation of a method to establish a pregnancy and the second implementation of a method to establish a pregnancy.

Methods for improved reproductive management of dairy cows are provided according to aspects of the present invention which include providing a database for reproductive management including a plurality of database entries; providing a biological sample including leukocytes of a first cow, wherein the leukocytes are characterized by the fact that they were drawn from the first cow after the first cow underwent implementation of a method to establish pregnancy; fixing and permeabilizing the leukocytes; contacting the leukocytes with a binding agent specific for an interferon-induced analyte to be assayed, thereby producing a first fluorescently-labeled sample; analyzing the first fluorescently-labeled sample by flow cytometry, thereby obtaining new flow cytometry data for the first cow; and updating the database with the new flow cytometry data for the first cow, wherein each database entry of the plurality of database entries corresponds to a different database cow; each database entry includes database information that is relevant to either cow interferon signaling or estrus cycle when assessed in relation to other database information; the database information includes: (1) a unique identifier for the database cow of the database entry, (2) the species of the database cow, (3) the breed of the database cow, (4) the geographic location of the database cow, (5) the age or birth date of the database cow, (6) date(s) of attempts to establish pregnancy in the database cow, (7) historical flow cytometry data obtained after attempts to establish pregnancy in the database cow, and (8) confirmed pregnancies of and/or births by the database cow; and the database allows the determination of whether a cow is open based at least in part on historical flow cytometry data and new flow cytometry data.

Methods for improved reproductive management of dairy cows are provided according to aspects of the present invention which include providing a second biological sample including leukocytes of a second cow, wherein the leukocytes of the second cow are characterized by the fact that they were drawn from the second cow after the second cow underwent an initial method to establish pregnancy; fixing and permeabilizing the leukocytes of the second cow; contacting the leukocytes of the second cow with the binding agent, thereby producing a second fluorescently-labeled sample; analyzing the second fluorescently-labeled sample by flow cytometry, thereby obtaining new flow cytometry data for the second cow; determining whether the second cow is open based on the database and the new flow cytometry data for the second cow, thereby obtaining a determination that the second cow is open or not open; and implementing a subsequent method to establish pregnancy in the second cow within 5 days of obtaining the determination if the determination is that the second cow is open, wherein if the database were not updated with the new flow cytometry data for the first cow, then (a) the determination would not have been that the second cow was open, and (b) the subsequent method to establish pregnancy in the second cow would not have been implemented within 5 days of the determination.

Methods for improved reproductive management of dairy cows are provided according to aspects of the present invention which include implementing a software algorithm after updating the database with the new flow cytometry data for the first cow, wherein the software algorithm is configured to minimize the probability of either a false positive determination or a false negative determination; and either the software algorithm is implemented as part of the determining step to obtain a determination that is specific to the second cow; or the software algorithm is implemented prior to the determining step, implementation of the software algorithm sets generally-applicable threshold values, and the determining step includes comparing the new flow cytometry data for the second cow and the database entry for the second cow with the generally-applicable threshold values.

Methods for improved reproductive management of dairy cows are provided according to aspects of the present invention wherein the database information of each database entry further includes one or more of: (7) date(s) on which the database cow of the database entry was administered antibiotics, if any, (9) dates on which the database cow was administered vaccines, if any, (10) dates on which the database cow was administered a pharmaceutical agent other than antibiotics or vaccines, if any, (11) medical history of the database cow related to parasite infection, viral infection, bacterial infection, and/or trauma (12) body temperature measurements of the database cow, (13) diet of the database cow, (14) genetic information of the database cow, and (15) climate and/or weather information associated with the geographic location.

Methods for improved reproductive management using a remote tracking and computer environment are provided according to aspects of the present invention which include providing a sample collection container having a first unique identifier, wherein the first unique identifier is visually readable (e.g., an alphanumeric) or electronically readable (e.g., a barcode such as a QR code); inserting an electronically-identifiable sample from an electronically-identifiable cow into the sample collection container, wherein: (a) the electronically-identifiable cow is associated with a second unique identifier; (b) the second unique identifier is visually readable (e.g., an ear tag) or electronically readable (e.g., a RFID tag); (c) the electronically-identifiable cow is the first cow or the second cow, and the electronically-identifiable sample is the sample including leukocytes of the first cow or the second cow, respectively; and (d) the electronically-identifiable sample is electronically-identifiable because it is inserted into the sample collection container; electronically recording the first unique identifier and the second unique identifier onto mobile storage media of a mobile device, thereby generating updated mobile storage media; wirelessly transmitting the first unique identifier and the second unique identifier from the updated mobile storage media to fixed storage media of a testing site computer, thereby generating updated fixed storage media; assigning the electronically-identifiable sample to an assigned sample well at the testing site; electronically recording the assigned sample well onto the updated fixed storage media, thereby generating assigned fixed storage media; transferring the electronically-identifiable sample from the sample collection container to the assigned sample well; and performing a method for improved reproductive management of dairy cows as described herein at the testing site.

Methods for improved reproductive management using a remote tracking and computer environment are provided according to aspects of the present invention wherein wirelessly transmitting the first unique identifier and the second unique identifier from the updated mobile storage media to the fixed storage media includes wirelessly transmitting the first unique identifier and the second unique identifier from the updated mobile storage media to remote storage media of a remote server, thereby generating updated remote storage media; and wirelessly transmitting the first unique identifier and the second unique identifier from the updated remote storage media to the fixed storage media, thereby generating the updated fixed storage media.

### BRIEF DESCRIPTION OF THE DRAWINGS

FIG. 1 is a bar graph showing the cumulative percentage of cow pregnancies that are lost on various days following artificial insemination (AI);

FIG. 2 is a bar graph showing the percentage of cows that are pregnant on various days following artificial insemination (AI);

FIG. 3 schematically illustrates a system for implementing computer and software based methods as described herein, according to one or more embodiments shown and described herein:

FIG. 4A is a graph showing three flow cytometry traces that assess the performance of an anti-Mx1 antibody;

FIG. 4B is a graph showing three flow cytometry traces that assess the performance of an anti-Mx2 antibody:

FIG. 4C is a graph showing three flow cytometry traces that assess the performance of an anti-ISG15 antibody;

FIG. 5A is a graph showing two flow cytometry traces that assess the performance of an anti-Mx1 antibody:

FIG. 5B is a graph showing three flow cytometry traces that assess the performance of an anti-ISG15 antibody:

FIG. 5C is a graph showing two flow cytometry traces that assess the performance of an anti-ISG15 antibody;

FIG. 6A contains four graphs that each show two flow cytometry traces that assess the performance of an anti-ISG15 antibody at a different time point for each graph;

FIG. 6B contains four graphs that each show two flow cytometry traces that assess the performance of an anti-Mx2 antibody at a different time point for each graph;

FIG. 6C contains four graphs that each show two flow cytometry traces that assess the performance of an anti-Mx1 antibody at a different time point for each graph:

FIG. 6D contains four graphs that each show two flow cytometry traces that assess the performance of a mixture of anti-ISG15, anti-Mx2, and anti-Mx1 antibodies at a different time point for each graph;

FIG. 7A is a flow cytometry plot showing the gating of leukocytes drawn from a cow 20 days after insemination;

FIG. 7B is a graph showing two flow cytometry traces of the fluorescence of labelled leukocytes on day 14.5 and day 20.0 after insemination;

FIG. 8A is a flow cytometry plot showing the gating of leukocytes drawn from a cow 20 days after insemination;

FIG. 8B is a graph showing two flow cytometry traces of the fluorescence of labelled leukocytes on day 14.5 and day 20.0 after insemination;

FIG. 9A is a flow cytometry plot showing the gating of leukocytes drawn from a cow 20 days after insemination:

FIG. 9C is a graph showing two flow cytometry traces of the fluorescence of labelled leukocytes on day 14.5 and day 20.0 after insemination;

FIG. 10A contains nine graphs showing flow cytometry traces each obtained from 9 different non-pregnant dairy cows;

FIG. 10B contains a graph showing a flow cytometry trace obtained from pooling flow cytometry data from 10 different non-pregnant dairy cows; and

FIG. 10C contains a graph showing flow cytometry data obtained from pooling flow cytometry data from 10 different non-pregnant dairy cows and a flow cytometry gate for differentiating open and non-open cells based on the pooled historical flow cytometry data.

### DETAILED DESCRIPTION OF THE INVENTION

Various aspects of the present invention relate to determining whether a female ruminant ungulate is “open” earlier than currently possible and allowing the female ruminant ungulate to undergo one or more additional attempts to establish pregnancy at more frequent intervals to reduce reproductive culling. Ovulation can typically be induced in open cows, for example, by administering prostaglandin F2α to them, and then such cows can be reinseminated with a high probability of conception.

Various aspects of the present invention relate to the finding that flow cytometry can be used to reproducibly determine whether a female ruminant ungulate is open based on the expression of INFT stimulated genes (ISGs) such as interferon-stimulated gene 15 (ISG15), Mx1 protein, and Mx2 protein in leukocytes obtained from the subject.

Various aspects of the present invention relate to methods of improved reproductive management that allow a subject to undergo implementation of a method to establish pregnancy following the determination that the subject is open without resynchronizing ovulation of the subject.

Dairy cows and other female ruminant ungulates are typically inseminated to give birth once every 12-14 months and the breeding period typically lasts 120-140 days. During the breeding period, the estrus cycle of a subject can be synchronized using hormones to induce ovulation, and then the subject is inseminated or implanted with an embryo.

The phrases “implementation of a method to establish pregnancy”, “attempt to establish pregnancy”, and similar phrases used herein refer to procedures implemented by a human to artificially inseminate a subject, impregnate the subject by embryo transfer or produce natural insemination of a subject (i.e., bringing two animals together for natural insemination of the subject by coitus).

The terms “synchronization” and “resynchronization” refer to both estrus synchronization and ovulation synchronization, which are processes of treating a female ruminant ungulate to ovulate at a precise time. The terms “synchronization” and “resynchronization” refer to such processes designed to grow and ovulate follicles and either inseminate a subject at a time to achieve a high probability of conception or transfer an embryo into a subject at a time to achieve a high probability of implantation and viability. Ovulation synchronization can be accomplished, for example in dairy cows, by administering gonadotropin releasing hormone (GnRH) seven days prior to an injection of prostaglandin F2α. This is followed in dairy cattle 56 hours later by a second injection of GnRH and insemination 16 hours later. Similar synchronization protocols can be used in other female ruminant ungulates.

If a cow becomes pregnant, then the pregnancy can be determined at about 28-30 days after insemination, for example, by detecting plasma pregnancy-specific protein B (PSPB aka PAG). The pregnancy of a cow cannot be reliably determined prior to about 28-30 days after conception because these analytes are not expressed in sufficient quantities until 28-30 days of pregnancy. Furthermore, many cows will lose existing pregnancies within 30 days of conception (see. e.g., FIG. 1). Up to 40% of cows that are pregnant after 21 days of gestation, for example, will lose their pregnancies by day 42.

As an example, if a synchronized and inseminated cow does not carry an embryo, then the cow is referred to as “open”, and, if not currently in estrus, the cow must be resynchronized and re-inseminated, otherwise it may be culled from the herd for infertility. Resynchronization typically involves the 10-day hormone protocol including GnRH and prostaglandin F2α described above and commonly termed Ovsynch 56. The cycle of insemination, pregnancy testing, and resynchronization takes about 38-45 days, which means that dairy cows can only be inseminated unsuccessfully 4-5 times before reaching a period where the farmer is compelled to cull the cow for infertility. Methods according to aspects of the present invention allow more attempts to establish pregnancy before this culling decision is made.

The unsuccessful implementation of a method to establish pregnancy in a female ruminant ungulate results in both the expense of reduced milk production and increases the probability of culling the subject, thereby ending its profitability. Likewise, in the beef industry that depends on a 12 month calving interval, after about 3 attempts to establish pregnancy, the farmer is faced with the decision to cull an open subject.

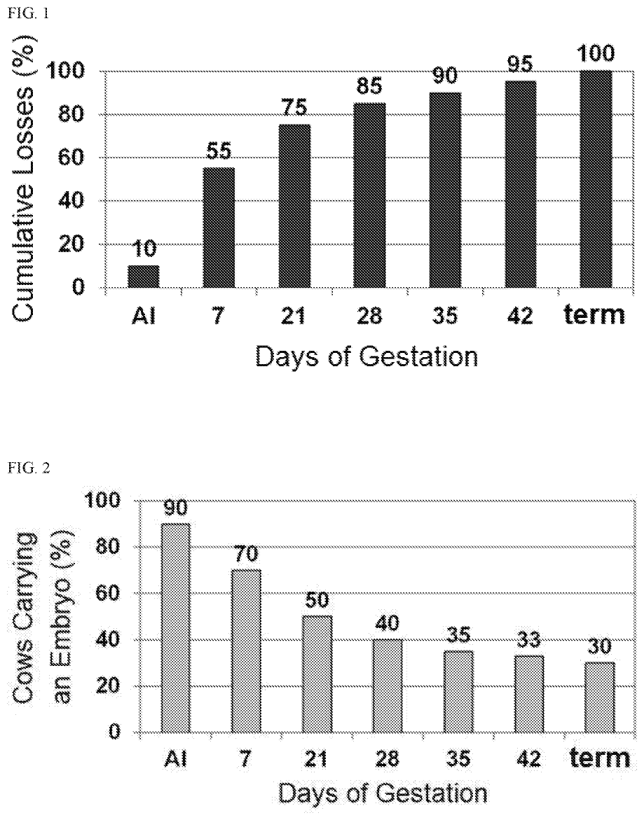

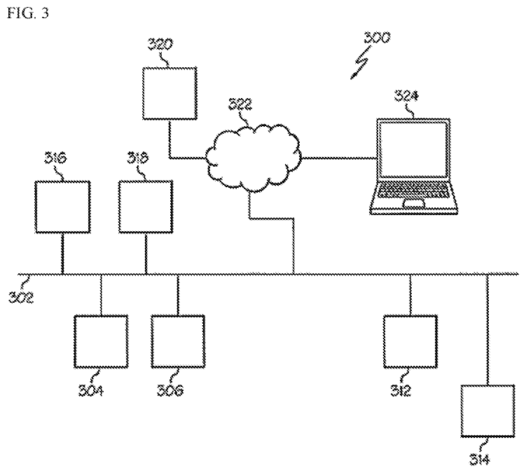

The detection of an open female ruminant ungulate within 3 weeks of an attempt to establish pregnancy allows for an earlier additional re-insemination or embryo-transfer attempt, thereby allowing increased attempts to establish a pregnancy before reaching that culling decision point. Notably, if a cow is open, the cow can be induced to ovulate, for example, by administering prostaglandin F2α to the cow at about 20 days following and unsuccessful insemination or about 13 days following unsuccessful embryo transfer. It is estimated that greater than 50% of inseminated cows are open at about 18-20 days after insemination (see. e.g., FIG. 2). The early identification of these subjects allows for re-insemination at 21-23 days thereby reducing the insemination interval by more than 10 days, saving the dairy farmer the cost of reduced milk production and culling the cow. Attempts to impregnate cows by embryo transfer display similar failure rates, and the early identification of open subjects similarly speeds additional embryo transfer attempts thereby reducing the interval between attempts to establish a pregnancy and increasing the average profitability of a herd of female ruminant ungulates.

Various aspects of the inventive methods described herein allow for the insemination of an ovulating female ruminant ungulate such as a cow, twice in 23 days, which was not previously feasible in many commercial dairies, particularly those that do not conduct heat detection. Various aspects of the inventive methods described herein allow for the embryo transfer of a female ruminant ungulate receptive to embryo transfer such as a cow, twice in 30 days, which was not previously feasible in many commercial farms, particularly those that do not conduct heat detection.

Various aspects of the present invention include methods of improved reproductive management of a female ruminant ungulate. The female ruminant ungulate can be, for example, a cow, sheep, goat, deer, elk, American buffalo, or water buffalo. The terms “subject” and “female ruminant ungulate” are used synonymously herein.

Methods are provided according to aspects of the present invention which include obtaining a biological sample from a subject, wherein the biological sample includes leukocytes. The biological sample can be “obtained” either directly from the subject, e.g., by drawing blood, or indirectly, e.g., by receiving the biological sample from a courier or by mail. The term “drawn” as used herein refers to obtaining leukocytes (or a sample containing leukocytes) directly from a subject, e.g., by drawing blood or milk directly from the subject, and thus, the scope of the term “draw” falls within the scope of the term “obtain”. The terms “obtain”, “obtained”, “obtaining”, etc. as used herein characterize the nature of a biological sample, when grammatically allowed by context, and thus, the phrase “leukocytes were obtained from a cow”, for example, discloses both (1) leukocytes characterized in that the leukocytes were obtained (i.e., drawn) from a cow, which may be used in various methods described herein, and (2) the act of obtaining (i.e., drawing) leukocytes from a cow, which may generally be included as a step in various methods described herein.

The terms “biological sample” and “sample” are used interchangeably herein, and these terms refer to a composition that includes leukocytes obtained from a female ruminant ungulate. A biological sample can include 10 to 10⁹ leukocytes, such as about 100 to about 10⁸ leukocytes, about 1,000 to about 10⁷ leukocytes, or about 10,000 to about 1,000,000 leukocytes, 100 μl of blood, for example, contains about 1,000,000 leukocytes, which is sufficient for the accurate determination of whether a female ruminant ungulate is open or not open. Fewer than 1,000,000 leukocytes can be analyzed for the accurate determination of whether a female ruminant ungulate is open or not open according to aspects of the present invention, such as about 1,000 leukocytes to about 100,000 leukocytes or such as about 1,000 leukocytes to about 10,000 leukocytes.

The analysis of a single leukocyte by flow cytometry is theoretically sufficient to accurately determine that a female ruminant ungulate is not open, e.g., if the single leukocyte contains a high concentration of an interferon-induced analyte. The minimum number of leukocytes required to determine whether a female ruminant ungulate is open or not open depends upon the standard operating procedure for an analysis, e.g., a lab may choose to reject samples that do not contain a specific number of leukocytes or to provide a determination with the caveat that a sample did not pass quality standards to support the certainty of a determination. In practice, the presence of very few leukocytes in a sample typically indicates that the sample has been mishandled, and a lab can nevertheless analyze the sample and provide a determination, if meaningful, along with a statement that the sample did not pass quality controls.

The leukocytes can optionally be peripheral blood leukocytes. According to various aspects of the present invention, the leukocytes can be obtained from blood or milk. The leukocytes can be either fixed and/or permeabilized, or a method can optionally include fixing and/or permeabilizing the leukocytes.

Methods are provided according to aspects of the present invention which include obtaining a biological sample from a female ruminant ungulate. Methods are provided according to aspects of the present invention which include obtaining a biological sample from a female ruminant ungulate, wherein the biological sample includes leukocytes, and the leukocytes were obtained from the female ruminant ungulate after implementing a method to establish a pregnancy in the female ruminant ungulate. Methods are provided according to aspects of the present invention which include obtaining a biological sample from a female ruminant ungulate, wherein the biological sample includes leukocytes, and the leukocytes were obtained from the female ruminant ungulate after insemination of the female ruminant ungulate. Methods are provided according to aspects of the present invention which include obtaining a biological sample from a female ruminant ungulate, wherein the biological sample includes leukocytes, and the leukocytes were obtained from the female ruminant ungulate after transferring an embryo in the female ruminant ungulate, a common assisted reproductive technology termed embryo transfer (ET).

Methods are provided according to aspects of the present invention which include obtaining a biological sample from a cow. Methods are provided according to aspects of the present invention which include obtaining a biological sample from a cow, wherein the biological sample includes leukocytes, and the leukocytes were obtained from the cow 15 to 25 days after insemination of the cow such as 17 to 21 days, 18 to 20 days, or 15, 16, 17, 18, 19, 20, 21, 22, 23, 24, or 25 days after insemination of the cow. Methods are provided according to aspects of the present invention which include obtaining a biological sample from a cow, wherein the biological sample includes leukocytes, and the leukocytes were obtained from the cow 8 to 19 days after transfer of an embryo into the cow such as 10 to 15 days, 11 to 14 days, or 8, 9, 10, 11, 12, 13, 14, 15, 16, 17, 18, or 19 days after the transfer of an embryo.

Methods are provided according to aspects of the present invention which include obtaining a biological sample from a female sheep. Methods are provided according to aspects of the present invention which include obtaining a biological sample from a female sheep, wherein the biological sample includes leukocytes, and the leukocytes were obtained from the sheep 12 to 22 days after insemination of the female sheep such as 13 to 15 days or 12, 13, 14, 15, 16, 17, 18, 19, 20, 21, or 22 days after insemination of the female sheep. Methods are provided according to aspects of the present invention which include obtaining a biological sample from a female sheep, wherein the biological sample includes leukocytes, and the leukocytes were obtained from the sheep 5 to 15 days after transfer of an embryo into the sheep such as 6 to 10 days, 6 to 8 days, or 5, 6, 7, 8, 9, 10, 11, 12, 13, 14, or 15 days after the transfer of an embryo.

Methods are provided according to aspects of the present invention which include obtaining a biological sample from a female goat. Methods are provided according to aspects of the present invention which include obtaining a biological sample from a female goat, wherein the biological sample includes leukocytes, and the leukocytes were obtained from the female goat 13 to 23 days after insemination of the female goat such as 14 to 18 days, 15 to 17 days, or 17 or 13, 14, 15, 16, 17, 18, 19, 20, 21, 22, or 23 days after insemination of the female goat. Methods are provided according to aspects of the present invention which include obtaining a biological sample from a female goat, wherein the biological sample includes leukocytes, and the leukocytes were obtained from the goat 6 to 18 days after transfer of an embryo into the female goat such as 7 to 13 days, 8 to 12 days, or 6, 7, 8, 9, 10, 11, 12, 13, 14, 15, 16, 17, or 18 days after the transfer of an embryo.

The foregoing dates for obtaining leukocytes from a cow, sheep, or goat that have undergone implementation of a method to establish a pregnancy such as natural or artificial insemination or embryo transfer are also generally applicable to other female ruminant ungulates based on the estrus cycle of the species and breed. Subjects that undergo embryo transfer rather than insemination typically receive the embryo transfer 5 to 8 days into an estrus cycle (i.e., after ovulation), such as 5 to 7 days or 5, 6, 7, or 8 days, and thus, a sample is typically obtained about 5 to 8 days earlier following embryo transfer relative to insemination (although the count of days following the synchronization of a subject is typically the same within a species or breed regardless of the method to establish a pregnancy).

The term “insemination” as used herein refers to both artificial insemination and natural insemination. Methods are provided according to aspects of the present invention wherein insemination refers only to artificial insemination.

The precise volume of a biological sample is not particularly limiting so long as the biological sample contains leukocytes. A sample can include, for example, about 1 μL to about 1 L of blood such as about 10 μL to about 100 mL, about 50 μL to about 50 mL, about 50 μL to about 10 mL, about 50 μL to about 5 mL, about 50 μL to about 1 mL, about 50 μL to about 500 μL, or about 75 μL to about 250 μL. Methods are provided according to aspects of the present invention which include obtaining 0.1-0.2 mL blood of a subject. Blood can optionally be combined with a fixative, e.g., a biological sample can include both blood and a fixative or a biological sample can be fixed blood.

Methods are provided according to aspects of the present invention which include isolating leukocytes from a blood or milk sample. Isolating leukocytes optionally includes centrifugation, e.g., to isolate the leukocyte-enriched buffy coat blood fraction from other components of a blood sample, or differential lysis of erythrocytes. e.g., by incubation in buffer containing about 155 mM NH₄Cl, about 12 mM NaHCO₃, and about 0.1 mM ethylenediamine tetraacetate. Leukocytes can also be isolated from other cells by flow cytometry (e.g., the leukocytes can be physically isolated from other cells by flow cytometry, and leukocyte flow cytometry data can optionally be electronically isolated from other cell flow cytometry data using a flow cytometry gating strategy).

A biological sample can be obtained from blood, milk, or other secretions, although the precise nature of the biological sample is not particularly limiting so long as the biological sample contains leukocytes that express interferon-induced analytes following a conception event.

A biological sample is typically suitable for analysis by flow cytometry, e.g., after fixing and/or permeabilizing the leukocytes of the sample (e.g., if the leukocytes are not fixed and/or permeabilized already), and after contacting the leukocytes of the sample with a fluorescently-labeled binding agent or a binding agent and a fluorescently-labeled probe that specifically binds the binding agent (e.g., if the leukocytes have not already been contacted with a binding agent).

Two or more biological samples can be obtained and analyzed. The two or more biological samples can be obtained at the same time or at different times from a subject. The two or more biological samples can be obtained from the same subject or from two or more subjects.

Methods are provided according to aspects of the present invention which include obtaining a second biological sample from a subject. A second biological sample is a biological sample as the term “biological sample” is used herein.

A second biological sample according to aspects of the present invention can be from a subject that has undergone implementation of a method to establish pregnancy at least twice, for example, wherein a first biological sample was obtained between a first and second attempt to establish pregnancy, analysis of the first biological sample determined that the subject was open, and the second biological sample is obtained after a second attempt to establish pregnancy (i.e., the second biological sample is obtained on a day that is after the day on which the first biological sample was obtained such as about 20-30 days after, e.g., for a cow). The first and second attempt to establish pregnancy can occur, for example, during consecutive estrus cycles. A second biological sample can therefore be used to determine whether a subject remains open after the second attempt to establish pregnancy.

A second biological sample can be from a subject that has been inseminated at least twice, for example, wherein a first biological sample was obtained between a first and second insemination, analysis of the first biological sample determined that the subject was open, and the second biological sample is obtained after a second insemination (i.e., the second biological sample is obtained on a day that is after the day on which the biological sample was obtained such as about 20-30 days after, e.g., for a cow). The first and second insemination can occur, for example, during consecutive estrus cycles. A second biological sample can therefore be used to determine whether a subject remains open after at least two rounds of insemination.

A second biological sample can be from a subject that has undergone embryo transfer at least twice, for example, wherein a first biological sample was obtained between a first and second embryo transfer, analysis of the first biological sample determined that the subject was open, and the second biological sample is obtained after a second embryo transfer (i.e., the second biological sample is obtained on a day that is after the day on which the first biological sample was obtained such as about 20-30 days after, e.g., for a cow). The first and second embryo transfer can occur, for example, during consecutive estrus cycles. A second biological sample can therefore be used to determine whether a subject remains open after at least two embryo transfers.

A second biological sample can be from a subject that has previously conceived and given birth, for example, wherein a biological sample was obtained between the previous conception and the birth at a time point allowing for the determination of whether the subject was open or not open, a flow cytometry analysis was performed on the biological sample to determine whether the subject was open or not open, and the second biological sample is obtained after a subsequent attempt to establish pregnancy, e.g., to determine whether the subject is open or not open following the subsequent attempt to establish pregnancy. The flow cytometry analysis of the biological sample can therefore optionally be used to establish a baseline or threshold for the flow cytometry analysis of the second biological sample.

Methods are provided according to aspects of the present invention which include obtaining a plurality of biological samples. Different samples of a plurality of biological samples can be obtained from different subjects. Each biological sample of a plurality of biological samples can be obtained from a different subject that underwent an attempt to establish pregnancy, for example, and each biological sample can be analyzed by flow cytometry to obtain flow cytometry data and/or to determine whether a subject is open or not open. Such methods can be used to determine whether one or more subjects of a plurality of subjects are open or not open. The subjects can optionally be of the same species or breed, same geographic location, and/or same herd, etc. For example, a plurality of biological samples can be obtained from a herd of dairy cows to determine whether individual dairy cows of the herd of dairy cows are open or not open. Such methods can also be used either to populate a database for reproductive management as described herein and/or to establish baselines or thresholds for a species or breed, geographic location, herd, etc., respectively, for subsequent use in analyzing flow cytometry data, e.g., to improve the accuracy and/or precision of subsequent analyses including a determination that a subject is open or not open.

Methods are provided according to aspects of the present invention which include obtaining a plurality of biological samples. Different samples of a plurality of biological samples can be obtained from different subjects. Each biological sample of a plurality of biological samples can be obtained from a different inseminated subject, for example, and each biological sample can be analyzed by flow cytometry to obtain flow cytometry data and/or to determine whether an inseminated subject is open or not open. Such methods can be used to determine whether one or more inseminated subjects of a plurality of subjects are open or not open. The inseminated subjects can optionally be of the same species or breed, same geographic location, and/or same herd, etc. For example, a plurality of biological samples can be obtained from a herd of dairy cows to determine whether individual inseminated dairy cows of the herd of dairy cows are open or not open. Such methods can also be used either to populate a database for reproductive management as described herein and/or to establish baselines or thresholds for a species or breed, geographic location, herd, etc., respectively, for subsequent use in analyzing flow cytometry data, e.g., to improve the accuracy and/or precision of subsequent analyses including a determination that a subject is open or not open.

Methods are provided according to aspects of the present invention which include obtaining a plurality of biological samples. Different samples of a plurality of biological samples can be obtained from different subjects. Each biological sample of a plurality of biological samples can be obtained from a different subject that underwent embryo transfer, for example, and each biological sample can be analyzed by flow cytometry to obtain flow cytometry data and/or to determine whether a subject that underwent embryo transfer is open or not open. Such methods can be used to determine whether one or more subjects that underwent embryo transfer of a plurality of subjects are open or not open. The subjects that underwent embryo transfer can optionally be of the same species or breed, same geographic location, and/or same herd, etc. For example, a plurality of biological samples can be obtained from a herd of dairy cows to determine whether individual dairy cows that underwent embryo transfer of the herd of dairy cows are open or not open. Such methods can also be used either to populate a database for reproductive management as described herein and/or to establish baselines or thresholds for a species or breed, geographic location, herd, etc., respectively, for subsequent use in analyzing flow cytometry data, e.g., to improve the accuracy and/or precision of subsequent analyses including a determination that a subject is open or not open.

Different samples of a plurality of biological samples can be obtained from the same subject. Two or more biological samples of a plurality of biological samples (or all biological samples of a plurality) can be obtained from the same subject, e.g., during different breeding periods, following different inseminations and/or embryo transfers, on different days following the same insemination or embryo transfer, on the same day following the same insemination or embryo transfer, and/or prior to an insemination or embryo transfer. Biological samples obtained from the same subject can be used to determine baselines or thresholds for the subject and/or similar subjects, e.g., to improve the accuracy and/or precision of subsequent analyses including a determination that a subject is open or not open.

A first biological sample and a second biological sample of a plurality of biological samples can optionally be obtained from the same subject wherein the first biological sample and the second biological sample are drawn on different days, e.g., during the same estrus cycle. For example, the second biological sample can be drawn on a day after the first biological sample is drawn. A first biological sample of the plurality of biological samples can be obtained within +/−0, 1, 2, 3, 4, 5, 6, 7, 8, or 9 days of the date of insemination of a subject to establish a baseline or threshold for flow cytometry analysis, and a second biological ample of the plurality can be obtained 12, 13, 14, 15, 16, 17, 18, 19, 20, 21, 22, 23, 24, or 25 days after the insemination to determine whether the subject remains open after the insemination. Similarly, a first biological sample of a plurality can be obtained 12, 13, 14, 15, 16, 17, 18, 19, 20, 21, 22, 23, 24, or 25 days after a first insemination of a subject during a first year in which the first insemination resulted in conception, pregnancy, and birth, and a second biological sample of the plurality can be obtained 12, 13, 14, 15, 16, 17, 18, 19, 20, 21, 22, 23, 24, or 25 days after a second insemination of the subject during a second year, e.g., wherein the flow cytometry analysis of the first and second biological samples is used to generate historical and new flow cytometry data, respectively, to determine whether the subject remains open after the second insemination based on the historical and new flow cytometry data.

A first biological sample of the plurality of biological samples can be obtained within +/−0, 1, 2, 3, 4, 5, 6, or 7 days of the date of embryo transfer of a subject to establish a baseline or threshold for flow cytometry analysis, and a second biological sample of the plurality can be obtained 8, 9, 10, 11, 12, 13, 14, 15, 16, 17, 18, 19, or 20 days after the embryo transfer to determine whether the subject remains open after the embryo transfer. Similarly, a first biological sample of a plurality can be obtained 8, 9, 10, 11, 12, 13, 14, 15, 16, 17, 18, 19, or 20 days after a first embryo transfer of a subject during a first year in which the first embryo transfer resulted in pregnancy and birth, and a second biological sample of the plurality can be obtained 8, 9, 10, 11, 12, 13, 14, 15, 16, 17, 18, 19, or 20 days after a second embryo transfer of the subject during a second year, e.g., wherein the flow cytometry analysis of the first and second biological samples is used to generate historical and new flow cytometry data, respectively, to determine whether the subject remains open after the second embryo transfer based on the historical and new flow cytometry data.

A first biological sample of biological samples can be obtained 12, 13, 14, 15, 16, 17, 18, 19, 20, 21, 22, 23, 24, or 25 days after a first insemination of a first subject in which the first insemination resulted in conception, pregnancy, and birth, and a second biological sample of the plurality can be obtained 12, 13, 14, 15, 16, 17, 18, 19, 20, 21, 22, 23, 24, or 25 days after a second insemination of a second subject to determine whether the second subject remains open after the second insemination, e.g., and the first subject and second subject preferably share similar characteristics such as species, breed, age, number of prior births, geographic location, herd, diet, and/or medical history, etc. A first biological sample of biological samples can be obtained 8, 9, 10, 11, 12, 13, 14, 15, 16, 17, 18, 19, or 20 days after a first embryo transfer of a first subject in which the first embryo transfer resulted in pregnancy and birth, and a second biological sample of the plurality can be obtained 8, 9, 10, 11, 12, 13, 14, 15, 16, 17, 18, 19, or 20 days after a second embryo transfer of a second subject to determine whether the second subject remains open after the second embryo transfer, e.g., and the first subject and second subject preferably share similar characteristics such as species, breed, age, number of prior births, geographic location, herd, diet, and/or medical history, etc. Information obtained from the flow cytometry analysis of a plurality of biological samples can be used to establish various baselines and/or thresholds as described herein and to populate a database as described herein. e.g., to improve the accuracy and/or precision of a determination that a subject is open or not open based on both new flow cytometry data and historical flow cytometry data.

Methods are provided according to aspects of the present invention which include contacting leukocytes or a sample containing leukocytes as described herein with a binding agent specific for an interferon-induced analyte. The binding agent can optionally be or include an antibody or an antigen-binding fragment of an antibody such as a fluorescently-labeled antibody or a fluorescently-labeled antigen-binding fragment thereof. The binding agent does not necessarily require a fluorescent label, however, as a secondary antibody can be used, for example, to fluorescently-label the binding agent.

The phrases “specific binding,” “specific for,” and grammatical equivalents as used herein in reference to binding of an antibody or antigen binding fragment refers to binding of the antibody or antigen binding fragment to the specified analyte without substantial binding to other peptides or proteins present in a biological sample to be assayed for presence of the specified analyte. It is understood by the ordinarily skilled artisan that specific binding refers to specific binding as determinable by use of appropriate controls to distinguish it from nonspecific binding.

Methods are provided according to aspects of the present invention which include contacting leukocytes or a sample containing leukocytes as described herein with a binding agent specific for an interferon-induced analyte, wherein the binding agent includes a fluorescent label.

Methods are provided according to aspects of the present invention which include contacting leukocytes or a sample containing leukocytes as described herein with a binding agent, wherein the binding agent includes a nucleic acid. Binding agents that include a nucleic acid can be useful to detect the mRNA of an interferon-induced analyte. The nucleic acid can optionally include a fluorescent label, e.g., for direct detection using flow cytometry.

Methods are provided according to aspects of the present invention which include contacting leukocytes or a sample containing leukocytes as described herein with a binding agent, wherein the binding agent is not fluorescently labeled. A method can further include, for example, contacting leukocytes or a sample containing leukocytes with a secondary agent such as a fluorescently-labeled secondary antibody, thereby allowing detection of the binding agent using flow cytometry.

Methods are provided according to aspects of the present invention which include producing a complex between a binding agent and an interferon-induced analyte, e.g., by contacting a biological sample with the binding agent. A complex is typically a non-covalent interaction between the antigen-binding region of an antibody (i.e., of a binding agent) and the interferon-induced analyte, which is an antigen recognized by the antigen-binding region. A complex can also be, for example, a non-covalent interaction between a nucleotide sequence of a binding agent and a nucleotide sequence of mRNA encoding an interferon-induced protein (e.g., wherein the mRNA is the interferon-induced analyte), e.g., wherein the nucleotide sequence of the binding agent and the nucleotide sequence of the mRNA display a high level of sequence complementarity such that the two sequences hybridize upon contacting the biological sample with the binding agent and the two sequences remain hybridized during subsequent analysis by flow cytometry. Methods are provided according to aspects of the present invention wherein the binding agent specifically binds mRNA or protein corresponding to an interferon-induced analyte. Methods are provided according to aspects of the present invention wherein the binding agent specifically binds to an interferon-induced analyte selected from the group consisting of interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR) or mRNA protein corresponding to an interferon-induced analyte selected from the group consisting of interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR). Methods are provided according to aspects of the present invention wherein the binding agent specifically binds mRNA or protein corresponding to ISG15, Mx1, Mx2, RTP4, OAS, and/or PKR.

The binding agent can be or include a monoclonal antibody, the antigen-binding portion of a monoclonal antibody, or a polyclonal antibody.

Antibodies, antigen-binding fragments, selection of appropriate antibodies or antigen binding fragments, and methods for their use and generation are known in the art, for instance, as described in Antibody Engineering, Kontermann, R. and Dübel, S. (Eds.). Springer, 2001; Harlow, E. and Lane, D., Antibodies: A Laboratory Manual, Cold Spring Harbor Laboratory Press, 1988; Ausubel, F. et al., (Eds.), Short Protocols in Molecular Biology, Wiley, 2002; J. D. Pound (Ed.) Immunochemical Protocols, Methods in Molecular Biology, Humana Press, 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975).

Methods are provided according to aspects of the present invention which include contacting leukocytes or a sample containing leukocytes as described herein with a plurality of binding agents. For example, the plurality of binding agents can include a binding agent specific for a first interferon-induced analyte (e.g., ISG15), a second interferon-induced analyte (e.g., Mx1), a third interferon-induced analyte (e.g., Mx2), a fourth interferon-induced analyte (e.g., RTP4), a fifth interferon-induced analyte (e.g., OAS), and/or a sixth interferon-induced analyte (e.g., PKR).

A plurality of binding agents can include 2, 3, 4, 5, or 6 binding agents that specifically bind protein corresponding to ISG15, Mx1, Mx2, RTP4, OAS, and/or PKR. A plurality of binding agents can include 2, 3, 4, 5, or 6 antibodies that specifically bind an antigen of ISG15, Mx1, Mx2, RTP4, OAS, and/or PKR. A plurality of binding agents can include 2, 3, 4, 5, or 6 binding agents that specifically bind mRNA corresponding to ISG15, Mx1, Mx2, RTP4, OAS, and/or PKR.

Methods are provided according to aspects of the present invention which include contacting leukocytes or a sample containing leukocytes with a plurality of probes. Each probe of a plurality of probes is typically used to directly or indirectly fluorescently label leukocytes for analysis by flow cytometry. A probe including a fluorescent label is an example of direct fluorescent labeling. A probe lacking a fluorescent label (e.g., a primary antibody) and therefore requiring a fluorescently-labeled second probe (e.g., a secondary antibody) is an example of indirect fluorescent labeling.

A plurality of probes can optionally include at least one probe that is a secondary antibody, which is typically a fluorescently-labeled monoclonal or polyclonal antibody that specifically binds the Fc region of a primary antibody. A fluorescently-labeled anti-mouse IgG antibody is an example of a secondary antibody that can be used in combination with mouse primary antibodies. Secondary antibodies include, for example, fluorescently-labeled antibodies that specifically bind a binding agent.

A plurality of probes can optionally include a probe that is a molecule that specifically binds double-stranded nucleic acids such as DNA. A probe that specifically binds DNA is a useful flow cytometry control to differentiate isolated, mononuclear cells, for example, from cell clumps. Molecules that specifically bind to double-stranded nucleic acids include intercalating agents generally and propidium iodide, ethidium bromide, and 4′,6-diamidino-2-phenylindole specifically. The choice of molecule that specifically binds double-stranded nucleic acids is typically selected based on the absorption and emission spectra of other fluorescently-labeled probes such that the excitation and emission of the fluorescently-labeled molecules in a biological sample are compatible.

A plurality of probes can optionally include a probe that binds a constitutively-expressed intracellular protein such as actin, tubulin, or glyceraldehyde-3-phosphate dehydrogenase, such as an anti-actin, anti-(beta-3) tubulin, or anti-glyceraldehyde-3-phosphate dehydrogenase antibody. A probe that binds a constitutively-expressed intracellular protein is a useful flow cytometry control to determine whether leukocytes are fixed and permeabilized.

Leukocytes or a sample containing leukocytes can be contacted with individual probes of a plurality of probes either simultaneously or consecutively. For example, either leukocytes or a sample containing leukocytes can be contacted with a primary antibody, the leukocytes or sample containing leukocytes can be washed, and then the leukocytes or sample can be contacted with a secondary antibody, which is an example of consecutive steps. Two or more probes of a plurality of probes can nevertheless be mixed such that the two or more probes of the plurality of probes are simultaneously contacted with leukocytes or a sample containing leukocytes.

Methods are provided according to aspects of the present invention which include contacting leukocytes or a sample containing leukocytes with a fixative such as contacting leukocytes or the sample containing leukocytes with a fixative solution. For example, a method can include fixing leukocytes. A method can optionally include combining blood and a fixative solution.

Fixatives include formaldehyde, paraformaldehyde, glutaraldehyde, and proprietary reagents such as Cytofix™ (BD Biosciences) and TransFix® (Cytomark). The choice of fixative is not particularly limiting so long as the fixative is compatible with the binding agent (e.g., the fixative does not denature an interferon-induced analyte epitope that is targeted by a binding agent that is an antibody) and the fixative is compatible with flow cytometry analysis (e.g., the fixative allows the leukocytes of a biological sample to be resuspended in solution).

In certain aspects, methods are provided herein in which the leukocytes and/or sample is not contacted with a fixative or fixative solution, e.g., because the leukocytes and/or sample is obtained from a third party who has already fixed the leukocytes and/or sample.

Methods are provided according to aspects of the present invention which include contacting leukocytes or a sample containing leukocytes with a permeabilization agent. Methods are provided according to aspects of the present invention which include permeabilizing leukocytes as described herein. Leukocytes or a sample containing leukocytes can be contacted with a permeabilization agent, for example, at the same time that the leukocytes or sample is contacted with a fixative, or the permeabilization agent and the fixative can be the same agent. Leukocytes can optionally be fixed and permeabilized at the same time. Leukocytes are nevertheless typically fixed and then permeabilized in separate steps, and the leukocytes can optionally already be fixed when a biological sample is obtained. Leukocytes are typically either fixed and then permeabilized or fixed and permeabilized at the same time rather than permeabilizing first and then fixing because permeabilization without fixation can cause the leukocytes to leak intracellular components including interferon-induced analyte.

Permeabilization agents include detergents such as saponin (e.g., 1% saponin) and Triton X-100 (e.g., 1% Triton X-100), alcohol such as ethanol, and proprietary reagents such as Perm/Wash™ (BD Biosciences).

Methods are provided according to aspects of the present invention which include contacting leukocytes or a sample containing leukocytes with a blocking agent. The blocking agent can include, for example, milk, casein, serum (e.g., fetal bovine serum and/or goat serum, such as about 1% to 4% fetal bovine serum and/or about 1% to 20% goat serum), or a purified, protein-containing fraction of any one of the foregoing.

Methods are provided according to aspects of the present invention which include contacting leukocytes or a sample containing leukocytes with a cryopreservative such as glycerol (e.g., 10% glycerol) or dimethyl sulfoxide (DMSO, e.g., 10% DMSO). Cryopreservative are generally unnecessary if leukocytes or a sample containing leukocytes are already fixed, but leukocytes or a sample containing leukocytes can nevertheless be contacted with a cryopreservative either before or after fixing leukocytes to further protect the leukocytes during storage at temperatures less than or equal to 0° C.

Methods are provided according to aspects of the present invention which include detecting a complex between a binding agent and an interferon-induced analyte using flow cytometry, thereby allowing for a determination of whether a subject is open or not open.

The precise flow cytometry protocol is not particularly limiting. Leukocytes are typically gated based on forward scatter and side scatter. Leukocytes can optionally be gated based on relative abundance of a double-stranded nucleic acid stain, e.g., to distinguish isolated, mononuclear cells from clumps of cells. Leukocytes can optionally be gated based on the fluorescence intensity of a probe that specifically labels a constitutively-expressed intracellular marker protein such as actin, tubulin, or glyceraldehyde-3-phosphate dehydrogenase, e.g., to distinguish permeabilized cells from non-permeabilized cells. Leukocytes can optionally be gated based on the fluorescence intensity of a probe that specifically labels lymphocytes, monocytes, basophils, neutrophils, eosinophils, or granulocytes, e.g., to distinguish lymphocytes, monocytes, basophils, neutrophils, eosinophils, or granulocytes from other cells of a biological sample. Leukocytes are typically gated based on the fluorescence intensity of a fluorescently-labeled binding agent specific for an interferon-induced analyte such as ISG15, Mx1, or Mx2 or a fluorescently-labeled probe that specifically binds an unlabeled binding agent specific for an interferon-induced analyte.

Methods are provided according to aspects of the present invention which include detecting a second complex between a binding agent and an interferon-induced analyte using flow cytometry, thereby allowing for a determination of whether a subject is open or not open, i.e., following a previous determination that the subject was open.

Detecting a complex between a binding agent and an interferon-induced analyte using flow cytometry typically produces new flow cytometry data, which can be used to determine whether a subject is open or not open (e.g., with a given accuracy).

Methods are provided according to aspects of the present invention which include either determining that a subject is open or determining that a subject is not open. This analysis differs significantly from a pregnancy test, for example, because a subject that is not open will not necessarily give birth. A subject that is not open may have, for example, a pregnancy that is not viable. Up to 40% of cows that carry an embryo at 21 days of gestation will lose the embryo, and thus, no method can currently diagnose a viable pregnancy in cows at or prior to 21 days of gestation. Subjects that are not open include pregnant subjects, certain subjects that were pregnant but that lost their pregnancies, and subjects that are pregnant with pregnancies that are not viable.

For example, cows that are not open typically cannot be productively re-inseminated (i.e. with a reasonable chance to produce a conception) within 18-20 days of their previous insemination because a conception event terminated the estrus cycle. Cows that are open can typically be productively re-inseminated (i.e. with a reasonable chance to produce a conception) at 18-20 days after a first, unsuccessful, insemination attempt based on the predictable presence of an ovarian follicle suitable for ovulation at this time (e.g., that occurs at about 20 days after the start of an estrus cycle in cows). Cows that are open can similarly typically undergo embryo transfer at 20-30 days after a previous embryo transfer.

Methods are provided according to aspects of the present invention which include communicating to the owner of a female ruminant ungulate (or an agent thereof) a determination that the female ruminant ungulate is open or not open. Methods are provided according to aspects of the present invention which include inseminating or transferring an embryo into a subject after determining that the subject is open.

Methods are provided according to aspects of the present invention which include providing a database for reproductive management. The database can be, for example a storage or database 314 as described in greater detail with respect to FIG. 3. A database includes a plurality of database entries. Each database entry of the plurality of database entries typically corresponds to a different female ruminant ungulate, which is referred as a “database female ruminant ungulate” or “database subject” such as a “database cow”.

A database entry includes database information that is relevant to either interferon signaling or estrus status when assessed in relation to other database information. Database information can include, for example, one or more of (1) a unique identifier for the database subject of the database entry, (2) the species of database subject, (3) the breed of database subject, (4) the geographic location of the database subject, (5) the age or birthday of the database subject, (6) date(s) of insemination and/or embryo transfer of the database subject, (7) historical flow cytometry data obtained after one or more inseminations and/or embryo transfers of the database subject. (8) confirmed pregnancies of and/or births by the database subject, (9) date(s) on which the database subject was administered antibiotics, if any, (10) dates on which the database subject was administered vaccines, if any, (11) dates on which the database subject was administered a pharmaceutical agent other than antibiotics or vaccines, if any, (12) medical history of the database subject related to parasite infection, viral infection, bacterial infection, and/or trauma, (13) body temperature measurements of the database subject, (14) diet of the database subject, (15) genetic information of the database subject, and (16) climate and/or weather information associated with the geographic location.

A database allows the determination of whether a subject is open or not open based at least in part on historical flow cytometry data and new flow cytometry data.

Methods are provided according to aspects of the present invention which include updating a database entry of a plurality of database entries with new flow cytometry data and/or with other database information.

Methods are provided according to aspects of the present invention which include identifying a baseline interferon-induced analyte expression level. The term “baseline” can refer to the expression level of an interferon-induced analyte as determined by flow cytometry on leukocytes obtained from one or more subjects that are open although the precise definition of the term baseline is dependent on how a baseline is determined and utilized. A baseline typically refers to a percentage of cells that pass an ISG-related fluorescence gate and all other relevant flow cytometry gates (e.g., forward scatter and side scatter). Methods are provided according to aspects of the present invention which include comparing new flow cytometry data to a baseline, e.g., thereby allowing for a determination of whether a subject is open or not open.

The precise method of setting a flow cytometry gate is not particularly limiting. A flow cytometry gate for ISG-related fluorescence events can be set, for example, as a multiple relative to either low-fluorescing cells (e.g., the 10% lowest fluorescing cells), a flow cytometry standard (e.g., immortalized, species-matched leukocytes), or a control sample from a known open subject. The multiple can be, for example, about 2, 3, 4, 5, 6, 7, 8, 9, or 10.

Based on a given flow cytometry gate for ISG-related fluorescence events, a baseline can optionally refer to a percentage of gated cells. A baseline is set to minimize either the probability of making a false positive determination (e.g., a determination that a subject is open when the subject is actually not open) or the probability of making a false negative determination (e.g., a determination that a subject is not open when the subject is actually open). For example, a baseline can be the maximum percentage of cells that are gated on ISG-related fluorescence events (and that pass all other flow cytometry gates) that nevertheless allows for a determination that a subject is open with a given accuracy (e.g., at least 99.9999% accuracy, which would allow for 1 false positive in 1 million samples). Similarly, a baseline can be the minimum percentage of cells that are gated on ISG-related fluorescence events (and that pass all other flow cytometry gates) that nevertheless allows for a determination that a subject is not open with a given accuracy (e.g., at least 99.9% accuracy, which would allow for 1 false negative in 1000 samples). A baseline can be specific to an individual subject, multiple subjects of a specific breed that have given birth a given number of times (e.g., 0 births, 1 birth, or >1 births), or multiple subjects of a specific breed in a specific geographic location (e.g., a herd), for example, although the precise delineation of a baseline is dependent upon (1) the nature of the biological sample(s) to be analyzed relative to the baseline and (2) the nature of the data that underpins the baseline (e.g., the size of a dataset of historical flow cytometry data, which is relevant to the accuracy of a determination relative to the baseline).

Various aspects of the inventive methods allow for the identification of trends in a database using a computational method to increase the accuracy of determining whether a subject is open or not open. A trend can be identified, for example, by a trend identifier component 312 in system 300 as described herein with respect to FIG. 3. Various factors affect the baseline levels of interferon-induced analyte expression in subjects including: the species and breed of subject; the geographic location of a subject including the climate and weather of the location; pathogens that a subject has been exposed to including parasites, viruses, and bacteria as documented in the medical records of a specific subject or as documented for a specific herd or geographic location; medical history of a subject including temperature, vaccinations, antibiotic regimens, and other treatments; and number of previous births by a subject, if any.

Humans cannot efficiently identify trends in a database of disparate variables to determine how an aggregate of disparate variables affects the expression of a biomarker in a given subject. Software algorithms can efficiently perform numerous mathematical calculations to either (1) set a threshold value for a specific flow cytometry gate (e.g., as a percentage of cells that pass other gates including side scatter and forward scatter gates) and set a threshold value for determining that a specific subject or a specific type of subject is open or not open based on the specific flow cytometry gate, and/or (2) determine that a specific subject is either open or not open with a specific level of certainty.

Such software algorithms present an efficient and improved methodology to provide such threshold setting and/or calculated output in a real-time manner such that users across a network, which optionally can be a network 322 that can be a cloud computing-based network configuration as described herein with respect to FIG. 3, can have real-time access to such information as it is available to allow for a streamlined and improved technical solution to a technical problem of determining, for example, whether a subject is open or not open across multiple platforms with updated and accurate information that can be, for example, provided in real-time. Such software algorithms can optionally employ an artificial intelligence tool 316 and/or trend identifier component 312 as described in greater detail below with respect to FIG. 3 to apply machine learning to automatically update and adapt data utilized in the algorithms, which adapted data can take into account data derived from, for example, the trend identifier component 312.

For example, a software algorithm can be configured to set both a flow cytometry gate to detect cells having an elevated interferon-induced analyte concentration (e.g., based on a fluorescence intensity higher than baseline fluorescence intensity) and a threshold percentage of gated cells relative to ungated cells (e.g., that otherwise pass other flow cytometry gates such as forward scatter and side scatter gates) such that the threshold allows for the determination of whether a subject is open or not open with a given accuracy (e.g., at least 99.9% accuracy). The gate and threshold can be dependent on other database information such that different gates and thresholds are set for different subjects, for example, based on database information.

Similarly, a software algorithm can be configured to simply determine the probability that a subject is open or not open based on the database information, e.g., as in a Bayesian probability. The software algorithm can therefore allow the determination that a subject is not open (e.g., if the probability that the subject is open is less than 50%, for example), that a subject is open (e.g., if the probability that the subject is open is greater than 99.999%, for example), or that the analysis is inconclusive (e.g., if the probability that the subject is open is 50% to 99.999%, for example).

The term “software algorithm” refers to any mathematical operation that allows for the calculation (i.e., determination) that a subject is either open or not open based on historical data for the same subject and/or similar subjects including (a) information that correlates (or inversely correlates) with whether the same subject and/or similar subjects were open or not open at a previous time point and (b) the actual status of the same subject and/or similar subjects as being open or not open at the previous time point. The precise nature of the software algorithm is not particularly limiting, and the software algorithm can include, for example, Monte Carlo, simulated annealing, hidden Markov model, and/or neural network components. The software algorithm can be, for example, an artificial intelligence software algorithm.

A software algorithm can optionally include instructions including a step of receiving data for at least one subject. The data can optionally include historical data for a specific subject and/or other subjects as set forth herein. The software algorithm can optionally include a step of associating one or more unique identifiers with one or more subjects and/or samples as described herein. The software algorithm can optionally include a step of setting threshold values such as, for example, a threshold for a flow cytometry gate and a threshold for making a determination based on a relative number of gated cells as described further below. The software algorithm can optionally include another step of determining whether the at least one subject is open or not open at least partially based on the received data and/or threshold values. The software algorithm can optionally also include a step of determining a probability that one or more subjects is open or not open as described herein. The software algorithm can optionally further include a step of identifying one or more trends at least partially based on the received data and/or determinations. Additionally, the software algorithm can optionally provide instructions to generate and/or display reports on select portions of the received data, the threshold values, the determinations, or combinations thereof.

The purpose of the software algorithm is to identify dominant factors that can affect the accuracy of a flow cytometry analysis. Differences in vaccination strategies can affect interferon-signaling in different subjects, for example, and in some cases, a recent vaccination can display a dominant effect on interferon-signaling that is relevant to determining whether a subject is open or not open. Software algorithms are typically superior to humans at adjusting a method of determining whether a subject is open or not open based in part on the vaccinations received by the subject and historical vaccination information for the same subject and/or for similar subjects, and an software algorithm can consider the historical vaccination information in light of other database information to optimize the accuracy of a determination.

Methods are provided according to aspects of the present invention which include implementing a software algorithm. The software algorithm can be implemented, for example, after updating a database with the new flow cytometry data.

The software algorithm is typically configured to minimize the probability of either a false positive determination or a false negative determination.

The software algorithm can optionally be implemented as part of a determining step to obtain a determination that is specific to a subject, e.g., as in a Bayesian probability.

The software algorithm can optionally be implemented prior to a determining step, wherein implementation of the software algorithm sets generally-applicable threshold values (such as a threshold for a flow cytometry gate and a threshold for a determination based on a relative number of gated cells), and the determining step includes comparing the new flow cytometry data for a subject and the database entry for the subject with the generally-applicable threshold values.

Methods are provided according to aspects of the present invention which include tracking of subjects, samples, and reproductive status. Various aspects of the inventive methods relate to improved reproductive management through the interrogation of samples by comparison of “open” and “not open” data in a computer environment.

Methods are provided according to aspects of the present invention which include providing a sample collection container having a first unique identifier. The first unique identifier is typically visually readable (e.g., an alphanumeric code) or electronically readable (e.g., a barcode such as a QR code).

Methods are provided according to aspects of the present invention which include inserting an electronically-identifiable sample into a sample collection chamber, wherein the electronically-identifiable sample is “electronically-identifiable” because it is inserted into the sample collection container. An electronically-identifiable sample is a biological sample as the term biological sample is defined herein.

An electronically-identifiable sample can be, for example, from an electronically-identifiable female ruminant ungulate such as an electronically-identifiable cow. An electronically-identifiable female ruminant ungulate is a subject that is associated with a second unique identifier. A second unique identifier can be visually readable (e.g., an ear tag) or electronically readable (e.g., a RFID tag). An electronically readable second unique identifier can be, for example, an RFID tag that is implanted in the subject such as under the skin of the subject.

Methods are provided according to aspects of the present invention which include recording a first unique identifier and/or a second unique identifier onto mobile storage media of a mobile device thereby generating updated mobile storage media. The term “storage media”, as used herein, refers to digital storage media such as a solid-state drive that is readable by a CPU when connected to the CPU through a wired connection interface such as an interface connector, integrated circuit, or USB port. As a non-limiting example, such storage media can include a memory component 306 as described in greater detail further below with respect to FIG. 3. Mobile storage media of a mobile device can be, for example, the solid-state drive of a cellphone, tablet computer, or laptop computer (or a hard disk drive of a laptop computer). The precise nature of the mobile storage media and mobile device is not particularly limiting. Methods are provided according to aspects of the present invention wherein the mobile device is configured to read a barcode (e.g., the mobile device includes a barcode reader or camera) and/or the mobile device is configured to read a RFID tag. A mobile device is typically configured for wireless connectivity, e.g., the mobile device is configured to transmit and/or receive data over a cellular network, Wi-Fi network, or Bluetooth. The mobile device need not necessarily be configured for wireless connectivity, however.

Methods are provided according to aspects of the present invention which include wirelessly transmitting a first unique identifier and/or second unique identifier from updated mobile storage media to fixed storage media of a testing site computer thereby generating updated fixed storage media. Wirelessly transmitting refers to any electronic data transmission that relies on electromagnetic radiation as electromagnetic radiation is used in commercial mobile devices, such as mobile phones, including cellular data transmission, Wi-Fi data transmission, and Bluetooth data transmission. The fixed storage media of a testing site computer can be, for example, the solid-state drive or hard disk drive of a desktop computer, although the precise nature of the testing site computer is not particularly limiting.

Methods are provided according to aspects of the present invention wherein wirelessly transmitting the first unique identifier and/or the second unique identifier from the updated mobile storage media to the fixed storage media includes (1) wirelessly transmitting the first unique identifier and the second unique identifier from the updated mobile storage media to remote storage media of a remote server thereby generating updated remote storage media, and (2) wirelessly transmitting the first unique identifier and/or the second unique identifier from the updated remote storage media to the fixed storage media thereby generating the updated fixed storage media. The term “remote” means that the server is electronically accessible (e.g., via an internet connection) and that the geographic location of the server is not limiting. A remote server can optionally be owned and operated by a third party that specializes in electronic data management, e.g., to ensure data security and accessibility and/or to provide data curation services such as regular backups, although a remote server can be owned and operated, for example, by a laboratory that performs the flow cytometry methods described herein. A remote server can be a “cloud” server as the term “cloud server” is used in the art.

The first unique identifier and/or the second unique identifier can optionally be transmitted from mobile storage media to fixed storage media using (1) a first wireless-configured device that wirelessly transmits data including the first unique identifier and/or the second unique identifier and (2) a second wireless-configured device that wirelessly receives data including the first unique identifier and/or the second unique identifier from the first wireless-configured device, e.g., wherein the mobile device and/or testing site computer do not wirelessly transmit and/or wirelessly receive the data, respectively. Accordingly, wirelessly transmitting a first unique identifier and/or a second unique identifier from mobile storage media to fixed storage media does not necessarily require that a mobile device or testing site computer have wireless connectivity.

Methods are provided according to aspects of the present invention which include assigning the electronically-identifiable sample to an assigned sample well at the testing site and recording the assigned sample well onto the updated fixed storage media thereby generating assigned fixed storage media. Methods are provided according to aspects of the present invention which include transferring the electronically-identifiable sample from the sample collection container to the assigned sample well at the testing site. Assigning sample wells and recording assigned sample wells allows for the automated processing of an electronically-identifiable sample, e.g., such that an electronically-identifiable sample remains electronically-identifiable after transfer to a sample well, and the sample thereby remains cross-referenced to the subject from which the sample originated.

Referring to FIG. 3, a system 300 for implementing computer and software-based methods such as software algorithms as described herein is illustrated as being implemented along with using a graphical user interface (GUI) that is accessible at a user workstation (e.g., a computer 324), for example. The system 300 includes a communication path 302, one or more processors 304, a memory component 306, a trend identifier component 312, a storage or database 314, an artificial intelligence tool component 316, a network interface hardware 318, a network 322, a server 320, and at least one computer 324. The various components of the system 300 and the interaction thereof will be described in detail below.

While only one application server 320 and one user workstation computer 324 is illustrated, the system 300 can include multiple workstations and application servers containing one or more applications that can be located at geographically diverse locations across a plurality of industrial sites. The system 300 can optionally be implemented using a wide area network (WAN) or network 322, such as an intranet or the Internet, or other wired or wireless communication network that can optionally include a cloud computing-based network configuration. The workstation computer 324 can optionally include digital systems and other devices permitting connection to and navigation of the network. Other system 300 variations allowing for communication between various geographically diverse components are possible. The lines depicted in FIG. 3 indicate communication rather than physical connections between the various components.

As noted above, the system 300 includes the communication path 302. The communication path 302 can optionally be formed from any medium that is capable of transmitting a signal such as, for example, conductive wires, conductive traces, optical waveguides, or the like, or from a combination of mediums capable of transmitting signals. The communication path 302 communicatively couples the various components of the system 300. As used herein, the term “communicatively coupled” means that coupled components are capable of exchanging data signals with one another such as, for example, electrical signals via conductive medium, electromagnetic signals via air, optical signals via optical waveguides, and the like.

As noted above, the system 300 includes the processor 304. The processor 304 can be any device capable of executing machine readable instructions. Accordingly, the processor 304 can optionally be a controller, an integrated circuit, a microchip, a computer, or any other computing device. The processor 304 is communicatively coupled to the other components of the system 300 by the communication path 302. Accordingly, the communication path 302 can optionally communicatively couple any number of processors with one another, and allow the modules coupled to the communication path 302 to operate in a distributed computing environment. Specifically, each of the modules can operate as a node that can send and/or receive data.

As noted above, the system 300 includes the memory component 306 which is coupled to the communication path 302 and communicatively coupled to the processor 304. The memory component 306 can optionally be a non-transitory computer readable medium or non-transitory computer readable memory and can optionally be configured as a nonvolatile computer readable medium. The memory component 306 can optionally include RAM, ROM, flash memories, hard drives, or any device capable of storing machine readable instructions such that the machine readable instructions can be accessed and executed by the processor 304. The machine readable instructions can optionally include logic or algorithm(s), such as software algorithms as described herein, written in any programming language such as, for example, machine language that can be directly executed by the processor, or assembly language, object-oriented programming (OOP), scripting languages, microcode, etc., that can be compiled or assembled into machine readable instructions and stored on the memory component 306. Similarly, the machine readable instructions can also be written in a hardware description language (HDL), such as logic implemented via either a field-programmable gate array (FPGA) configuration or an application-specific integrated circuit (ASIC), or their equivalents. Accordingly, the methods described herein can be implemented in any conventional computer programming language, as pre-programmed hardware elements, or as a combination of hardware and software components. In embodiments, the system 300 can optionally include the processor 304 communicatively coupled to the memory component 306 that stores instructions that, when executed by the processor, cause the processor to perform one or more tool functions such as machine readable instructions execution as described herein.

Still referring to FIG. 3, as noted above, the system 300 includes the display such as a GUI on a screen of the computer 324 for providing visual output such as, for example, information, graphical reports, messages, or a combination thereof. The computer 324 can optionally include one or more computing devices across platforms, or can optionally be communicatively coupled to devices across platforms, such as mobile smart devices including smartphones, tablets, laptops, and/or the like. The display on the screen of the computer 324 is coupled to the communication path 302 and communicatively coupled to the processor 304. Accordingly, the communication path 302 communicatively couples the display to other modules of the system 300. The display can include any medium capable of transmitting an optical output such as, for example, a cathode ray tube, light emitting diodes, a liquid crystal display, a plasma display, or the like. Additionally, it is noted that the display or the computer 324 can include at least one of the processor 304 and the memory component 306. While the system 300 is illustrated as a single, integrated system in FIG. 3, in other embodiments, the systems can be independent systems.

The system 300 optionally includes the artificial intelligence tool component 316 for applying machine learning to one or more software algorithms such as those executed by the trend identifier component 312 to assist with integration of the system 300 with other tools as described above. The artificial intelligence tool component 316 and the trend identifier component 312 are coupled to the communication path 302 and communicatively coupled to the processor 304.

Data stored and manipulated in the system 300 as described herein is utilized by the artificial intelligence tool component 316, which is able to leverage a cloud computing-based network configuration (for example, referable to as “the cloud”), to apply Machine Learning and Artificial Intelligence. This learning can optionally create models that can be applied by the system 300, to make it more efficient and intelligent in execution. As an example and not a limitation, the artificial intelligence tool component 316 can optionally include components selected from the group consisting of an artificial intelligence engine, Bayesian inference engine, and a decision-making engine, and can optionally have an adaptive learning engine further including a deep neural network learning engine.

As will be described in further detail below, the processor 304 can optionally process the input signals received from the system modules and/or extract information from such signals.

The system 300 includes the network interface hardware 318 for communicatively coupling the system 300 with a computer network such as network 322. The network interface hardware 318 is coupled to the communication path 302 such that the communication path 302 communicatively couples the network interface hardware 318 to other modules of the system 300. The network interface hardware 318 can be any device capable of transmitting and/or receiving data via a wireless network. Accordingly, the network interface hardware 318 can include a communication transceiver for sending and/or receiving data according to any wireless communication standard. For example, the network interface hardware 318 can include a chipset (e.g., antenna, processors, machine readable instructions, etc.) to communicate over wired and/or wireless computer networks such as, for example, wireless fidelity (Wi-Fi), WiMax, Bluetooth, IrDA, Wireless USB, Z-Wave, ZigBee, or the like.

Still referring to FIG. 3, data from various applications running on computer 324 can be provided from the computer 324 to the system 300 via the network interface hardware 318. The computer 324 can be any device having hardware (e.g., chipsets, processors, memory, etc.) for communicatively coupling with the network interface hardware 318 and a network 322. Specifically, the computer 324 can include an input device having an antenna for communicating over one or more of the wireless computer networks described above.

The network 322 can include any wired and/or wireless network such as, for example, wide area networks, metropolitan area networks, the Internet, an Intranet, satellite networks, or the like. Accordingly, the network 322 can be utilized as a wireless access point by the computer 324 to access one or more servers (e.g., a server 320). The server 320 and any additional servers generally include processors, memory, and chipset for delivering resources via the network 322. Resources can include providing, for example, processing, storage, software, and information from the server 320 to the system 300 via the network 322. Additionally, the server 320 and any additional servers can share resources with one another over the network 322 such as, for example, via the wired portion of the network, the wireless portion of the network, or combinations thereof.

Embodiments of inventive compositions and methods are illustrated in the following examples. These examples are provided for illustrative purposes and are not considered limitations on the scope of inventive compositions and methods.

### EXEMPLIFICATION

### Example 1: Sample Preparation Via In Vitro Treatment for Analysis

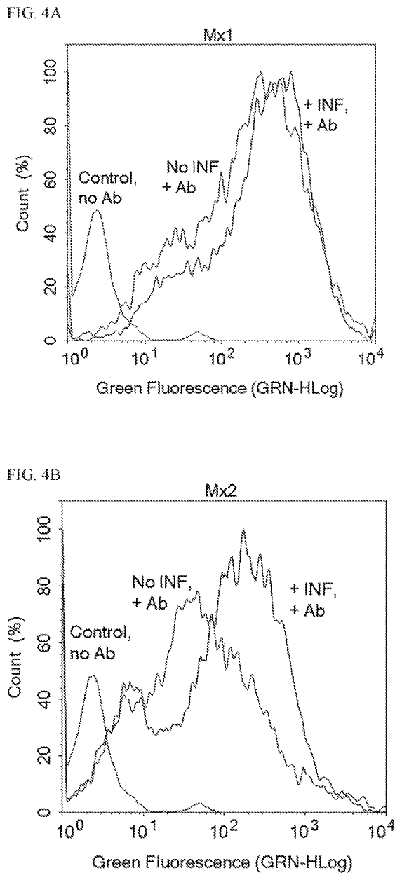

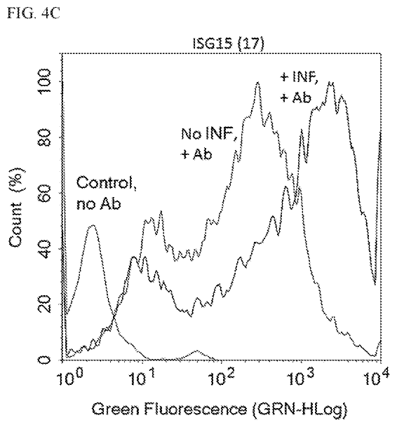

Bovine blood was drawn and fractionated by centrifugation. The buffy coat fraction was isolated from other blood fractions, and leukocytes of the buffy coat were incubated for 24 hours with or without INF-τ. The leukocytes were then fixed and frozen. Leukocytes were thawed and then contacted with 3 μg/mL of a primary antibody against either Mx1, Mx2, or ISG15 and then contacted with 5 μg/mL of a fluorescently-labeled secondary antibody. All incubations and wash steps were performed in Perm/Wash™ buffer (BD Biosciences). Cells were counted by flow cytometry. FIG. 4A-4C show flow cytometry plots for three antibodies.

FIG. 4A shows an overlay of three flow cytometry traces in which the x-axis corresponds to the relative fluorescence intensity of a secondary antibody and the y-axis corresponds to relative cell count. The left-most flow cytometry trace corresponds to a control sample that was not contacted with the secondary antibody. The two right-side flow cytometry traces correspond to two samples that were incubated for 24 hours with or without INFT and then contacted with both a primary anti-Mx1 antibody and a fluorescently-labeled secondary antibody. The sample that was incubated for 24 hours without INFT displayed a non-parametric distribution with a peak at about 200 on the x-axis. The sample that was incubated for 24 hours with INFT displayed a non-parametric distribution with a peak at about 900 on the x-axis.

FIG. 4B shows an overlay of three flow cytometry traces in which the x-axis corresponds to the relative fluorescence intensity of a secondary antibody and the y-axis corresponds to relative cell count. The left-most flow cytometry trace corresponds to a control sample that was not contacted with the secondary antibody. The two right-shifted flow cytometry traces correspond to two samples that were incubated for 24 hours with or without INFT and then contacted with both a primary anti-Mx2 antibody and a fluorescently-labeled secondary antibody. The sample that was not incubated for 24 hours with INFT displayed a non-parametric distribution with peak cell counts at about 6 and about 25 on the x-axis. The sample that was incubated for 24 hours with INFT displayed a non-parametric distribution with peaks at about 6 and about 60 on the x-axis and was shifted toward greater fluorescence than the controls.

FIG. 4C shows an overlay of three flow cytometry traces in which the x-axis corresponds to the relative fluorescence intensity of a secondary antibody and the y-axis corresponds to relative cell count. The left-most flow cytometry trace corresponds to a control sample that was not contacted with the secondary antibody. The two right-shifted flow cytometry traces correspond to two samples that were incubated for 24 hours with or without INFT and then contacted with both a primary anti-ISG15 antibody and a fluorescently-labeled secondary antibody. The sample that was not incubated for 24 hours with INFT displayed a non-parametric distribution with a peak cell counts at about 400 on the x-axis. The sample that was incubated for 24 hours with INFT displayed a non-parametric distribution with peaks at about 10 and about 2000 on the x-axis and was shifted toward greater fluorescence than the controls.

### Example 2. Sample Preparation Via In Vivo Treatment for Analysis

0.1 mL blood is obtained from a female ruminant ungulate, and the blood is added to a vial containing 4% paraformaldehyde as a fixative (or other fixative such as CytoFix™ (BD Biosciences) or TransFix® (Cytomark)). The fixation reaction is optionally quenched with borohydride depending on the nature of the fixative. The biological sample is then stored for 24 hours at 4° C. The cells of the biological sample are washed and then resuspended in Perm/Wash™ (BD Biosciences) or other detergent such as 1% saponin or 1% Triton X-100. The fixed, permeabilized cells are pelleted and resuspended in blocking buffer. The cells are either pelleted and resuspended in a solution containing a primary antibody (which targets an interferon-induced analyte) and then pelleted and resuspended in a solution containing a secondary antibody (which targets the primary antibody), or the cells are pelleted and resuspended in a solution containing a fluorescently-labeled primary antibody (which targets an interferon-induced analyte). The cells are optionally pelleted and resuspended in solutions(s) containing additional primary and/or secondary antibodies to detect additional targets including constitutively-expressed proteins (e.g., actin, tubulin), which serve as process controls, and/or additional interferon-induced analytes. The cells are pelleted and resuspended in phosphate buffered saline (PBS) containing 1 μg/mL propidium iodide (or other fluorescent nucleic acid stain, e.g., 4′,6-diamidino-2-phenylindole). Cells are analyzed by flow cytometry using gates for side scatter, forward scatter, propidium iodide, and the fluorescently-labelled antibody(s).

### Example 3: Identification of Suitable Blocking Buffers

A number of blocking buffers were screened according to a protocol similar to Example 1 including 12 blocking buffers provided by SurModics and various blocking buffers containing 4% fetal bovine serum, 1% w/v saponin, and 0-20% goat serum in PBS. The blocking buffers were screened in combination with two different anti-ISG15 antibodies and two different secondary antibodies. Two proprietary blocking buffers obtained from SurModics containing either casein base or milk base were found effective for use with the specific primary anti-ISG15 antibodies and the specific secondary antibodies. 10 proprietary blocking buffers were found ineffective for use with both cow leukocytes and the specific antibodies that were analyzed. Each blocking buffer containing 4% fetal bovine serum, 1% w/v saponin, and 0-20% goat serum in PBS was found effective for use with specific primary anti-ISG15 antibodies and secondary antibodies.

### Example 4: Identification of Suitable Anti-Interferon-Induced Analyte Antibodies

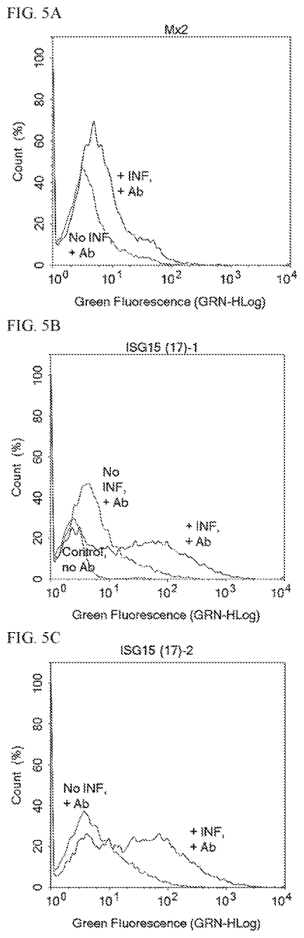

19 different anti-interferon-induced analyte antibodies were screened for compatibility with flow cytometry with cow leukocytes according to a protocol similar to Example 1. Three samples were analyzed by flow cytometry using each antibody including (1) a cow leukocyte sample to which no antibody was added, (2) a non-pregnant cow leukocyte sample to which a test antibody was added, and (3) an interferon-analog-stimulated cow leukocyte sample to which a test antibody was added. Some antibodies were incapable of differentiating unstimulated cow leukocytes from stimulated cow leukocytes. FIG. 5A, for example, depicts flow cytometry traces used to assess the performance of an anti-Mx1 protein antibody. FIG. 5A contains three flow cytometry traces. The trace on the left corresponds to a control sample to which no antibody was added. The two traces to the right of FIG. 5A correspond to unstimulated and interferon-analog-stimulated cow leukocyte samples, and flow cytometry using the specific anti-Mx1 protein antibody of FIG. 5A could not distinguish the two samples. FIGS. 5B and 5C depict flow cytometry traces used to assess the performance of two different anti-ISG15 antibodies. The left-most traces of FIGS. 5B and 5C correspond to control samples to which no antibody was added. The middle traces of FIGS. 5B and 5C correspond to unstimulated cow leukocyte samples. The right-most traces of FIGS. 5B and 5C correspond to interferon-analog-stimulated cow leukocyte samples. The anti-ISG15 antibodies assessed in FIGS. 5B and 5C suggest that these two antibodies can distinguish unstimulated an interferon-analog-stimulated cow leukocyte samples and that these antibodies may be particularly useful in the flow cytometry analysis of cow leukocytes to determine whether a cow is open or not open.

### Example 5: Time-Course of Interferon-Induced Analyte Expression after In Vivo Treatment with INFT

A cow was injected with INFT, and blood from the cow was drawn at various time points after injection. The buffy coat fractions of the blood were isolated from other blood fractions, and leukocytes of the buffy coat fractions were fixed and frozen. Fluorescently-labeled anti-ISG15, anti-Mx2, and anti-Mx1 antibodies, and a mixture of anti-ISG15, anti-Mx2, and anti-Mx1 antibodies were used to label the leukocytes for flow cytometry analysis. Propidium iodide (PI) was also added to samples, and a flow cytometry gate was set to differentiate mononucleated cells from other cells, cell clumps, and cell fragments. The body temperature of the cow was 101.7° F. immediately prior to the INFT injection, 104.5° F. at 4 hours after injection, and 101.8° F. at 24 hours after injection.

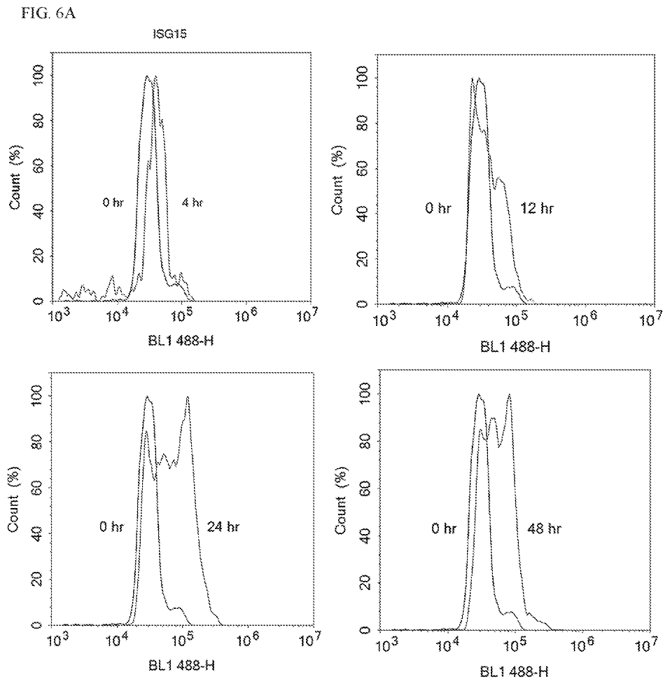

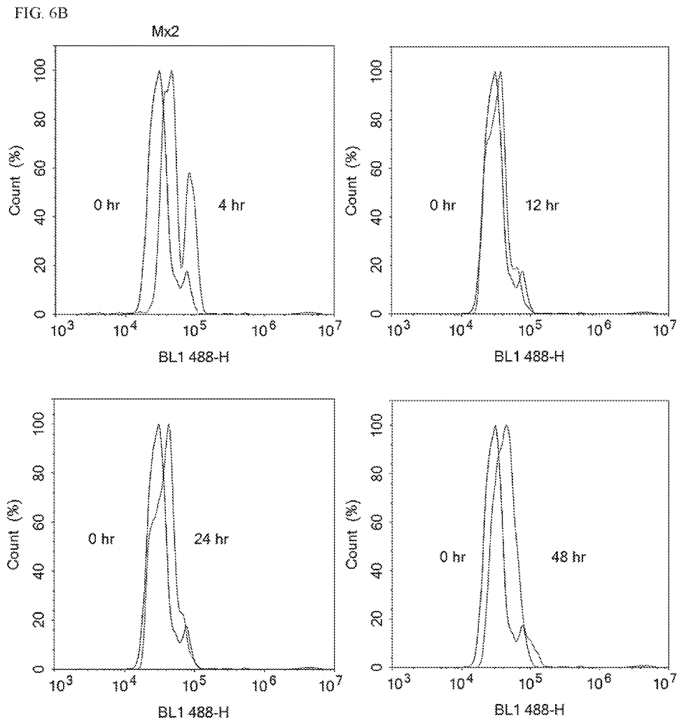

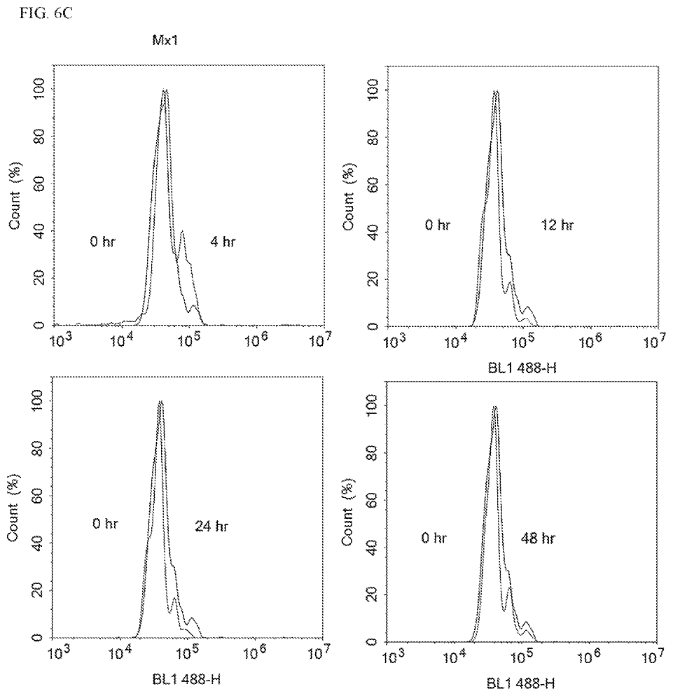

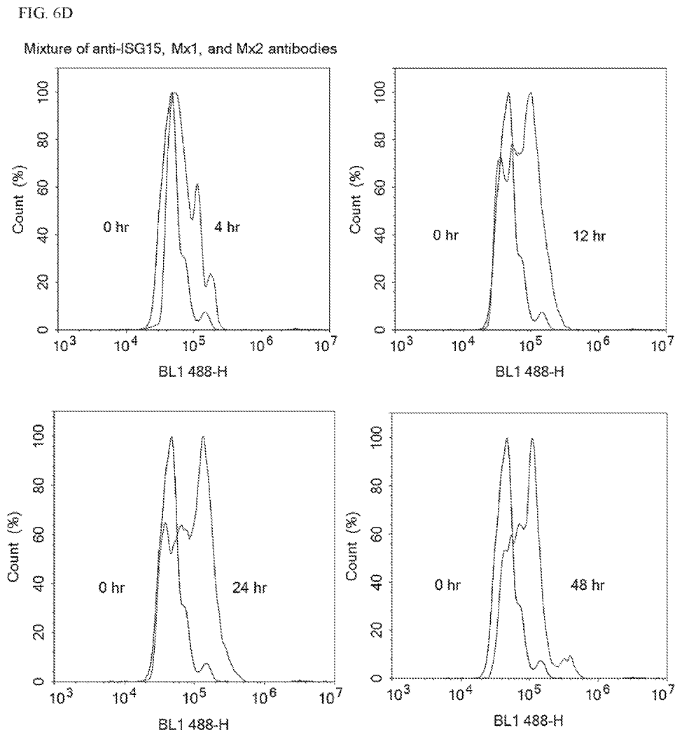

FIG. 6A contains four graphs containing two flow cytometry traces each. Each graph contains (1) a flow cytometry trace for leukocytes obtained 4, 12, 24, or 48 hours after a cow was injected with INFT and that were incubated with an anti-ISG15 antibody and (2) a flow cytometry trace for control leukocytes that were incubated with the same anti-ISG15 antibody. FIG. 6A shows that the anti-ISG15 antibody is capable at differentiating INFT stimulated leukocytes from control leukocytes at each time point, and the anti-ISG15 antibody was particularly useful at differentiating leukocytes 24-48 hours after INFT injection.

FIG. 6B contains four graphs containing two flow cytometry traces each. Each graph contains (1) a flow cytometry trace for leukocytes obtained 4, 12, 24, or 48 hours after a cow was injected with INFT and that were incubated with an anti-Mx2 antibody and (2) a flow cytometry trace for control leukocytes that were incubated with the same anti-Mx2 antibody. FIG. 6B shows that the anti-Mx2 antibody is capable at differentiating INFT stimulated leukocytes from control leukocytes at each time point, and the anti-Mx2 antibody was particularly useful at differentiating leukocytes 4 hours after INFT injection.

FIG. 6C contains four graphs containing two flow cytometry traces each. Each graph contains (1) a flow cytometry trace for leukocytes obtained 4, 12, 24, or 48 hours after a cow was injected with INFT and that were incubated with an anti-Mx1 antibody and (2) a flow cytometry trace for control leukocytes that were incubated with the same anti-Mx1 antibody. FIG. 6C shows that the anti-Mx1 antibody was particularly useful at differentiating leukocytes 4 hours after INFT injection.

FIG. 6D contains four graphs containing two flow cytometry traces each. Each graph contains (1) a flow cytometry trace for leukocytes obtained 4, 12, 24, or 48 hours after a cow was injected with INFT and that were incubated with a mixture of anti-ISG15, anti-Mx1, and anti-Mx2 antibodies and (2) a flow cytometry trace for control leukocytes that were incubated with the same mixture of antibodies. FIG. 6D shows that the mixture of antibodies is capable at differentiating INFT stimulated leukocytes from control leukocytes at each time point.

### Example 6: Analysis of Artificially Inseminated Female Ruminant Ungulate

A heifer was artificially inseminated at day 0. A blood sample was drawn from the heifer on day 14.5 after insemination. A second blood sample was drawn from the heifer on day 20 after insemination. The heifer was slaughtered, and the existence of an embryo was confirmed by flushing the uterus. The two samples were prepared for flow cytometry analysis using a method similar to the method disclosed in Example 2. Aliquots of the two samples were contacted with 5 μg/mL, 10 μg/mL, or 20 μg/mL of a binding agent mix including anti-Mx1, anti-Mx2, and anti-ISG15 IgG antibodies that had been fluorescently-labelled with an about 1:1 to 2:1 molar ratio of fluorescent label DyLight 488 to antibody. The samples were also contacted with propidium iodide to identify isolated mononuclear cells by flow cytometry.

The aliquots from the day 14.5 samples were used to set flow cytometry gates on DyLight 488 fluorescence intensity such that the 5% highest-fluorescence intensity cells of each aliquot of a day 14.5 sample that had passed every other flow cytometry gate were gated on DyLight 488 fluorescence intensity. The day 20 sample aliquots were then analyzed based on the DyLight 488 gates set for corresponding day 14.5 sample aliquots. 15.72%, 16.96%, and 21.35% of cells of the day 20 sample aliquots that were contacted with 5 μg/mL, 10 μg/mL, or 20 μg/mL of the binding agent mix, respectively, that passed every other flow cytometry gate were gated on DyLight 488 fluorescence intensity, and each percentage is significantly higher than the 5% of gated cells of the day 14.5 sample aliquots.

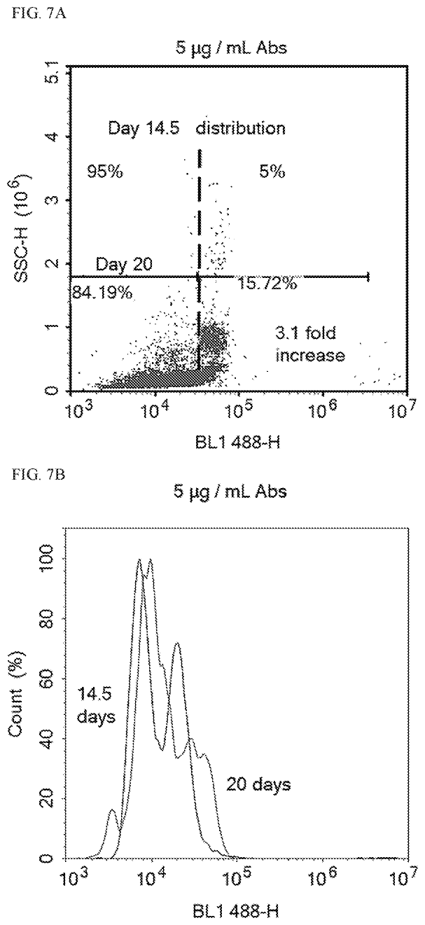

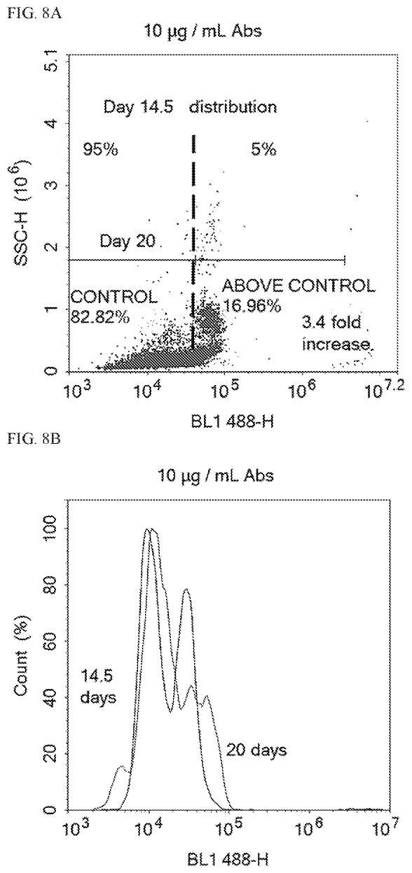

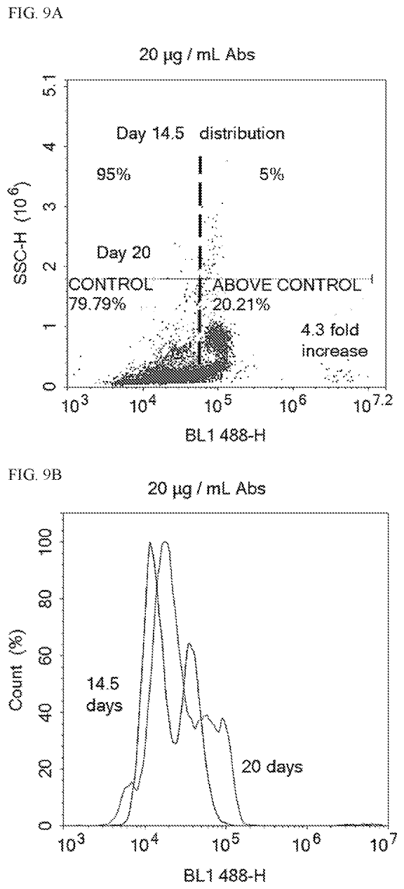

FIGS. 7A. 8A, and 9A are flow cytometry plots of day 20 sample aliquots that were contacted with 5 μg/mL, 10 μg/mL, or 20 μg/mL of the binding agent mix, respectively. FIG. 8A-8C depict DyLight 488 fluorescence intensity on the x-axis and side scatter on the y-axis. The vertical line in each of FIGS. 7A, 8A, and 9A depict the lower boundary of a flow cytometry gate that was set to gate the 5% highest-fluorescence intensity cells of a day 14.5 sample aliquot that was contacted with the same concentration of the binding agent mix as the day 20 sample aliquot for which results are shown in a given plot. FIGS. 7A. 8A, and 9A show that 15.72%, 16.96%, and 21.35% of cells of the day 20 sample aliquots were gated, respectively, with gates set according to the 5% highest-fluorescence intensity cells of a day 14.5 sample aliquots.

FIGS. 7B, 8B, and 9B are graphs that each contain two flow cytometry traces corresponding to a day 20 sample aliquot (denoted by x's) and a day 14.5 sample aliquot (denoted by o's). Each x-axis corresponds to DyLight 488 fluorescence intensity, and each y-axis corresponds to relative cell count. FIGS. 7B, 8B, and 9B correspond to sample aliquots that were contacted with 5 μg/mL, 10 μg/mL, or 20 μg/mL of the binding agent mix, respectively. The day 20 sample aliquots display a shift toward greater DyLight 488 fluorescence intensity relative to the day 14.5 sample aliquots, which was quantified using the gates described above.

### Example 7: Determining Baseline Flow Cytometry Gating Strategy by Bayesian Analysis

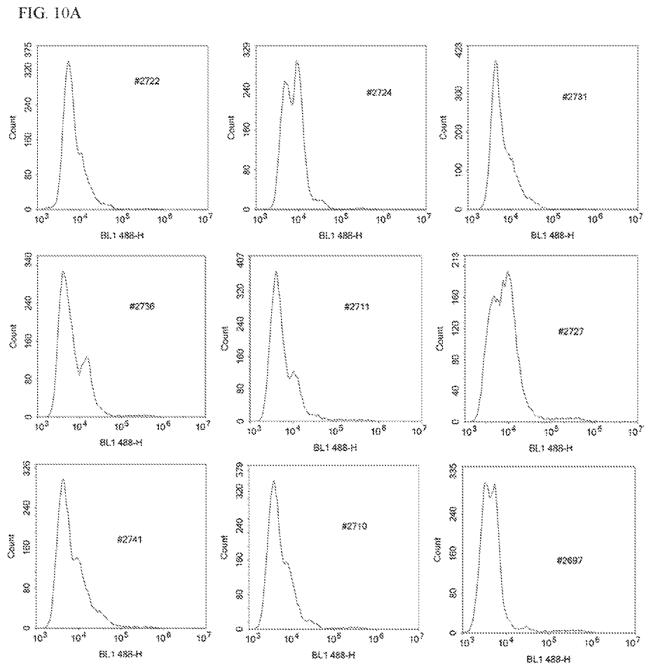

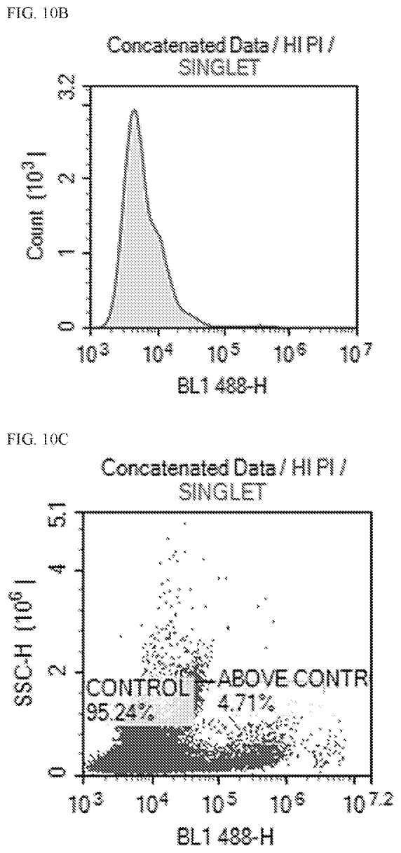

Ten cows were randomly selected from a dairy herd, and blood samples were obtained from each cow. Leukocytes of the blood samples were analyzed by flow cytometry using a protocol similar to that described in Example 2. The samples were treated or untreated with a mixture of antibodies directed against ISG15, Mx1, and Mx2 to fluorescently label leukocytes that express interferon-induced analytes. FIG. 10A depicts nine representative graphs of fluorescence intensity versus cell count from nine of the ten cows. The graphs of FIG. 10A include traces for leukocytes that were treated with the mixture of antibodies. FIG. 10B is a graph of fluorescence intensity versus cell count for pooled data for leukocytes of the ten cows that were contacted with the mixture of antibodies. FIG. 10C is a graph of fluorescence intensity versus side scatter for pooled data for leukocytes of the ten cows that were contacted with the mixture of antibodies. FIG. 10C also shows a flow cytometry gate that was set based on the pooled data. FIG. 10B-10C may be used to predict flow cytometry results for leukocyte samples from cows that are open, and FIGS. 10B and 10C may be used to set a flow cytometry gate to determine whether a cow is open or not open such as the gate depicted in FIG. 10C. The prediction of flow cytometry results for a typical open cow based on historical flow cytometry analysis as depicted in FIG. 10B-10C is a type of Bayesian analysis.

Items

Item 1. A method of improved reproductive management of a female ruminant ungulate, comprising: contacting a first biological sample with a first binding agent specific for an interferon-induced analyte to be assayed in the biological sample, thereby producing a first complex between the binding agent and the interferon-induced analyte, wherein the first biological sample comprises leukocytes of a female subject, and the subject is a female ruminant ungulate that underwent a first implementation of a method to establish pregnancy; and detecting the first complex using flow cytometry, thereby allowing for a determination of whether the subject is open or not open.

Item 2. The method of item 1, further comprising communicating to the owner of the subject or an agent thereof a determination that the subject is open or not open.

Item 3. The method of item 1 or 2, wherein the subject is a cow.

Item 4. The method of item 3, wherein the leukocytes are characterized by the fact that they were drawn from the cow 17 to 21 days after insemination of the cow or 10 to 16 days after embryo transfer into the cow.

Item 5. The method of item 4, wherein the leukocytes are characterized by the fact that they were drawn from the cow 18, 19, or 20 days after insemination of the cow or 11, 12, or 13 days after embryo transfer into the cow.

Item 6. The method of item 1 or 2, wherein the subject is a sheep.

Item 7. The method of item 6, wherein the leukocytes are characterized by the fact that they were drawn from the sheep 13 to 20 days after insemination of the sheep or 6 to 15 days after embryo transfer into the sheep.

Item 8. The method of item 7, wherein the leukocytes are characterized by the fact that they were drawn from the sheep 14, 15, or 16 days after insemination of the sheep or 7, 8, or 9 days after embryo transfer into the sheep.

Item 9. The method of any one of items 4-8, wherein it is determined that the female ruminant ungulate is open, further comprising: contacting a second biological sample with a second binding agent specific for the interferon-induced analyte to be assayed in the biological sample, thereby producing a second complex between the binding agent and the interferon-induced analyte; and detecting the second complex using flow cytometry, thereby allowing for a determination of whether the subject is open or not open, wherein: the second biological sample comprises leukocytes of the subject; the leukocytes of the second biological sample are characterized by the fact that they were drawn from the subject after the subject had undergone a second implementation of a method to establish pregnancy; the second implementation of the method to establish pregnancy occurred after detecting the first complex using flow cytometry; and the second implementation of the method to establish pregnancy occurred during a second estrus cycle immediately following a first estrus cycle during which the first implementation of the method to establish pregnancy occurred.

Item 10. The method of item 9, wherein: the first method to establish pregnancy is a first insemination and the second method to establish pregnancy is a second insemination; the second insemination occurred 21-24 days after the first insemination occurred; the leukocytes of the first biological sample are characterized by the fact that they were drawn from the subject during a first estrus of the subject; the first insemination occurred during the first estrus of the subject; the second insemination occurred during a second estrus of the subject; and the first estrus and the second estrus are estruses of consecutive estrus cycles.

Item 11. The method of item 9, wherein: the first method to establish pregnancy is a first embryo transfer and the second method to establish pregnancy is a second embryo transfer; the second embryo transfer occurred 21-24 days after the first embryo transfer occurred; the leukocytes of the first biological sample are characterized by the fact that they were drawn from the subject during a first estrus of the subject; the first embryo transfer occurred during the first estrus of the subject; the second embryo transfer occurred during a second estrus of the subject; and the first estrus and the second estrus are estruses of consecutive estrus cycles.

Item 12. The method of any one of items 1-11, wherein the first binding agent and second binding agent are specific for an interferon-induced analyte selected from the group consisting of: interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR) or mRNA corresponding to an interferon-induced analyte selected from the group consisting of interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR).

Item 13. The method of any one of items 1-12, wherein first binding agent and second binding agent are specific for the same interferon-induced analyte.

Item 14. The method of any one of items 1-13, wherein the binding agent comprises a monoclonal antibody, an antigen-binding portion of a monoclonal antibody, or a polyclonal antibody.

Item 15. The method of item 14, wherein the binding agent is an anti-ISG15 antibody, an anti-Mx1 antibody, an anti-Mx2 antibody, or a fluorescently-labeled analogue of any one of the foregoing.

Item 16. The method of any one of items 1-15, wherein the leukocytes of the biological sample are characterized by the fact that they are from about 0.1-0.2 mL blood that was drawn from the subject.

Item 17. The method of any one of items 1-16, wherein the leukocytes are peripheral blood leukocytes.

Item 18. The method of any one of items 1-15, wherein the leukocytes are characterized by the fact that they were obtained from milk or secretions from the reproductive tract.

Item 19. The method of any one of items 1-18, further comprising fixing and/or permeabilizing the leukocytes.

Item 20. The method of any one of items 1-19, further comprising contacting the leukocytes with a plurality of probes, wherein the plurality of probes comprises a probe that specifically labels double stranded nucleic acids, thereby allowing the identification of isolated, mononuclear cells by flow cytometry.

Item 21. The method of any one of items 1-20, further comprising contacting the leukocytes with a plurality of probes, wherein the plurality of probes comprises a probe that specifically labels a constitutively-expressed intracellular protein, thereby allowing the identification of permeabilized cells by flow cytometry.

Item 22. The method of any one of items 1-21, further comprising contacting the leukocytes with a plurality of probes, wherein the plurality of probes comprises a probe that specifically labels a protein expressed by lymphocytes, monocytes, eosinophils, or granulocytes, thereby allowing the identification of lymphocytes, monocytes, eosinophils, or granulocytes by flow cytometry.

Item 23. The method of any one of items 1-22, further comprising contacting the leukocytes with a blocking reagent selected from the group consisting of milk, casein, serum, and a purified protein fraction of milk, casein, or serum.

Item 24. A method of analyzing leukocytes of a female subject, comprising: providing a first biological sample comprising leukocytes of the subject, wherein the leukocytes are characterized by the fact that they were drawn from the subject after the subject underwent a first implementation of a first method to establish a pregnancy, and the subject is a female ruminant ungulate; fixing and permeabilizing the leukocytes; contacting the leukocytes with a binding agent, wherein the binding agent specifically labels a protein or mRNA corresponding to interferon stimulated gene 15 (ISG15). Mx1 protein (Mx1), Mx2 protein (Mx2), receptor transporter protein-4 (RTP4), 2′,5′ oligoadenylate synthetase (OAS), or protein kinase R (PKR), thereby allowing the identification of leukocytes that express one of the foregoing gene products by flow cytometry; analyzing the fluorescently-labeled sample by flow cytometry, thereby obtaining new flow cytometry data; and performing a second implementation of a second method to establish a pregnancy in the subject after analyzing the fluorescently-labeled sample if the new flow cytometry data determines that the subject is open.

Item 25. The method of item 24, wherein: the first method to establish a pregnancy was a first insemination; and the second method to establish a pregnancy comprises inseminating the subject 21-24 days after the first insemination.

Item 26. The method of item 24, wherein: the first method to establish a pregnancy was a first embryo transfer; and the second method to establish a pregnancy comprises transferring an embryo into the subject 20-30 days after the first embryo transfer.

Item 27. The method of any one of items 9-11 and 24-26, wherein the subject is not administered gonadotropin releasing hormone between the method to establish a pregnancy and the second method to establish a pregnancy.

Item 28. The method of any one of items 9-11 and 24-27, wherein the subject is administered prostaglandin F2α between the first implementation of the first method to establish a pregnancy and the second implementation of the second method to establish a pregnancy.

Item 29. A method for improved reproductive management of dairy cows, comprising: providing a database for reproductive management comprising a plurality of database entries; providing a biological sample comprising leukocytes of a first cow, wherein the leukocytes are characterized by the fact that they were drawn from the first cow after the first cow underwent a first implementation of a method to establish pregnancy; fixing and permeabilizing the leukocytes; contacting the leukocytes with a binding agent specific for an interferon-induced analyte to be assayed, thereby producing a first fluorescently-labeled sample; analyzing the first fluorescently-labeled sample by flow cytometry, thereby obtaining new flow cytometry data for the first cow; and updating the database with the new flow cytometry data for the first cow, wherein: each database entry of the plurality of database entries corresponds to a different database cow; each database entry comprises database information that is relevant to either cow interferon signaling or estrus cycle when assessed in relation to other database information; the database information comprises: (1) a unique identifier for the database cow of the database entry, (2) the species of the database cow, (3) the breed of the database cow, (4) the geographic location of the database cow. (5) the age or birth date of the database cow. (6) date(s) of attempts to establish pregnancy in the database cow, (7) historical flow cytometry data obtained after attempts to establish pregnancy in the database cow, and (8) confirmed pregnancies of and/or births by the database cow; and the database allows the determination of whether a cow is open based at least in part on historical flow cytometry data and new flow cytometry data.

Item 30. The method of item 29, further comprising: providing a second biological sample comprising leukocytes of a second cow, wherein the leukocytes of the second cow are characterized by the fact that they were drawn from the second cow after the second cow underwent an initial implementation of a method to establish pregnancy; fixing and permeabilizing the leukocytes of the second cow; contacting the leukocytes of the second cow with the binding agent, thereby producing a second fluorescently-labeled sample; analyzing the second fluorescently-labeled sample by flow cytometry, thereby obtaining new flow cytometry data for the second cow; determining whether the second cow is open based on the database and the new flow cytometry data for the second cow, thereby obtaining a determination that the second cow is open or not open; and implementing a subsequent method to establish pregnancy in the second cow within 5 days of obtaining the determination if the determination is that the second cow is open, wherein: if the database were not updated with the new flow cytometry data for the first cow, then (a) the determination would not have been that the second cow was open, and (b) the subsequent method to establish pregnancy in the second cow would not have been implemented within 5 days of the determination.

Item 31. The method of item 30, further comprising implementing a software algorithm after updating the database with the new flow cytometry data for the first cow, wherein: the software algorithm is configured to minimize the probability of either a false positive determination or a false negative determination; and either the software algorithm is implemented as part of the determining step to obtain a determination that is specific to the second cow; or the software algorithm is implemented prior to the determining step; implementation of the software algorithm sets generally-applicable threshold values; and the determining step comprises comparing the new flow cytometry data for the second cow and the database entry for the second cow with the generally-applicable threshold values.

Item 32. The method of any one of items 29-31, wherein the database information of each database entry further comprises one or more of: (7) date(s) on which the database cow of the database entry was administered antibiotics, if any, (9) dates on which the database cow was administered vaccines, if any, (10) dates on which the database cow was administered a pharmaceutical agent other than antibiotics or vaccines, if any, (11) medical history of the database cow related to parasite infection, viral infection, bacterial infection, and/or trauma (12) body temperature measurements of the database cow. (13) diet of the database cow, (14) genetic information of the database cow, and (15) climate and/or weather information associated with the geographic location.

Item 33. A method for improved reproductive management using a remote tracking and computer environment, comprising: providing a sample collection container having a first unique identifier, wherein the first unique identifier is visually readable (e.g., an alphanumeric) or electronically readable (e.g., a barcode such as a QR code); inserting an electronically-identifiable sample from an electronically-identifiable cow into the sample collection container, wherein: (a) the electronically-identifiable cow is associated with a second unique identifier; (b) the second unique identifier is visually readable (e.g., an ear tag) or electronically readable (e.g., a RFID tag); (c) the electronically-identifiable cow is the first cow or the second cow, and the electronically-identifiable sample is the sample comprising leukocytes of the first cow or the second cow, respectively; and (d) the electronically-identifiable sample is electronically-identifiable because it is inserted into the sample collection container; electronically recording the first unique identifier and the second unique identifier onto mobile storage media of a mobile device, thereby generating updated mobile storage media; wirelessly transmitting the first unique identifier and the second unique identifier from the updated mobile storage media to fixed storage media of a testing site computer, thereby generating updated fixed storage media; assigning the electronically-identifiable sample to an assigned sample well at the testing site; electronically recording the assigned sample well onto the updated fixed storage media, thereby generating assigned fixed storage media; transferring the electronically-identifiable sample from the sample collection container to the assigned sample well; and performing the method of any one of items 29-32 at the testing site.

Item 34. The method of item 33, wherein wirelessly transmitting the first unique identifier and the second unique identifier from the updated mobile storage media to the fixed storage media comprises: wirelessly transmitting the first unique identifier and the second unique identifier from the updated mobile storage media to remote storage media of a remote server, thereby generating updated remote storage media; and wirelessly transmitting the first unique identifier and the second unique identifier from the updated remote storage media to the fixed storage media, thereby generating the updated fixed storage media.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of improved reproductive management of a female ruminant ungulate, comprising:
contacting a first biological sample with a first binding agent specific for an interferon-induced analyte to be assayed in the biological sample, thereby producing a first complex between the binding agent and the interferon-induced analyte, wherein the first biological sample comprises leukocytes of a female subject, and the subject is a female ruminant ungulate that underwent a first implementation of a method to establish pregnancy; and
detecting the first complex using flow cytometry, thereby allowing for a determination of whether the subject is open or not open.

2. The method of claim 1, further comprising communicating to the owner of the subject or an agent thereof a determination that the subject is open or not open.

3. The method of claim 1, wherein the subject is a cow.

4. The method of claim 3, wherein the leukocytes are characterized by the fact that they were drawn from the cow 17 to 21 days after insemination of the cow or 10 to 16 days after embryo transfer into the cow.

5. The method of claim 4, wherein the leukocytes are characterized by the fact that they were drawn from the cow 18, 19, or 20 days after insemination of the cow or 11, 12, or 13 days after embryo transfer into the cow.

6. The method of claim 1, wherein the subject is a sheep.

7. The method of claim 6, wherein the leukocytes are characterized by the fact that they were drawn from the sheep 13 to 20 days after insemination of the sheep or 6 to 15 days after embryo transfer into the sheep.

8. The method of claim 7, wherein the leukocytes are characterized by the fact that they were drawn from the sheep 14, 15, or 16 days after insemination of the sheep or 7, 8, or 9 days after embryo transfer into the sheep.

9. The method of claim 4, wherein it is determined that the female ruminant ungulate is open, further comprising:
contacting a second biological sample with a second binding agent specific for the interferon-induced analyte to be assayed in the biological sample, thereby producing a second complex between the binding agent and the interferon-induced analyte; and
detecting the second complex using flow cytometry, thereby allowing for a determination of whether the subject is open or not open, wherein:
the second biological sample comprises leukocytes of the subject;
the leukocytes of the second biological sample are characterized by the fact that they were drawn from the subject after the subject had undergone a second implementation of a method to establish pregnancy;
the second implementation of the method to establish pregnancy occurred after detecting the first complex using flow cytometry; and
the second implementation of the method to establish pregnancy occurred during a second estrus cycle immediately following a first estrus cycle during which the first implementation of the method to establish pregnancy occurred.

10. The method of claim 9, wherein:
the first method to establish pregnancy is a first insemination and the second method to establish pregnancy is a second insemination;
the second insemination occurred 21-24 days after the first insemination occurred;
the leukocytes of the first biological sample are characterized by the fact that they were drawn from the subject during a first estrus of the subject;
the first insemination occurred during the first estrus of the subject;
the second insemination occurred during a second estrus of the subject; and
the first estrus and the second estrus are estruses of consecutive estrus cycles.

11. The method of claim 9, wherein:
the first method to establish pregnancy is a first embryo transfer and the second method to establish pregnancy is a second embryo transfer;
the second embryo transfer occurred 21-24 days after the first embryo transfer occurred;
the leukocytes of the first biological sample are characterized by the fact that they were drawn from the subject during a first estrus of the subject;
the first embryo transfer occurred during the first estrus of the subject;
the second embryo transfer occurred during a second estrus of the subject; and
the first estrus and the second estrus are estruses of consecutive estrus cycles.

12. The method of claim 1, wherein the first binding agent and second binding agent are specific for an interferon-induced analyte selected from the group consisting of: interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR) or mRNA corresponding to an interferon-induced analyte selected from the group consisting of interferon stimulated gene 15 (ISG15), Mx1 protein (Mx1), Mx2 protein (Mx2), receptor transporter protein-4 (RTP4), 2′,5′ oligoadenylate synthetase (OAS), and protein kinase R (PKR).

13.-23. (canceled)

24. A method of analyzing leukocytes of a female subject, comprising:
providing a first biological sample comprising leukocytes of the subject, wherein the leukocytes are characterized by the fact that they were drawn from the subject after the subject underwent a first implementation of a first method to establish a pregnancy, and the subject is a female ruminant ungulate;
fixing and permeabilizing the leukocytes;
contacting the leukocytes with a binding agent, wherein the binding agent specifically labels a protein or mRNA corresponding to interferon stimulated gene 15 (ISG15), Mx1 protein (Mx1), Mx2 protein (Mx2), receptor transporter protein-4 (RTP4), 2′,5′ oligoadenylate synthetase (OAS), or protein kinase R (PKR), thereby allowing the identification of leukocytes that express one of the foregoing gene products by flow cytometry;
analyzing the sample by flow cytometry, thereby obtaining new flow cytometry data; and
performing a second implementation of a second method to establish a pregnancy in the subject after analyzing the sample if the new flow cytometry data determines that the subject is open.

25. The method of claim 24, wherein:
the first method to establish a pregnancy was a first insemination; and
the second method to establish a pregnancy comprises inseminating the subject 21-24 days after the first insemination.

26. The method of claim 24, wherein:
the first method to establish a pregnancy was a first embryo transfer; and
the second method to establish a pregnancy comprises transferring an embryo into the subject 20-30 days after the first embryo transfer.

27. The method of claim 9, wherein the subject is not administered gonadotropin releasing hormone between the method to establish a pregnancy and the second method to establish a pregnancy.

28. The method of claim 9, wherein the subject is administered prostaglandin F2α between the first implementation of the first method to establish a pregnancy and the second implementation of the second method to establish a pregnancy.

29. A method for improved reproductive management of dairy cows, comprising:
providing a database for reproductive management comprising a plurality of database entries;
providing a biological sample comprising leukocytes of a first cow, wherein the leukocytes are characterized by the fact that they were drawn from the first cow after the first cow underwent a first implementation of a method to establish pregnancy;
fixing and permeabilizing the leukocytes;
contacting the leukocytes with a binding agent specific for an interferon-induced analyte to be assayed, thereby producing a first fluorescently-labeled sample;
analyzing the first fluorescently-labeled sample by flow cytometry, thereby obtaining new flow cytometry data for the first cow; and updating the database with the new flow cytometry data for the first cow, wherein:
each database entry of the plurality of database entries corresponds to a different database cow;
each database entry comprises database information that is relevant to either cow interferon signaling or estrus cycle when assessed in relation to other database information;
the database information comprises: (1) a unique identifier for the database cow of the database entry, (2) the species of the database cow, (3) the breed of the database cow, (4) the geographic location of the database cow, (5) the age or birth date of the database cow, (6) date(s) of attempts to establish pregnancy in the database cow, (7) historical flow cytometry data obtained after attempts to establish pregnancy in the database cow, and (8) confirmed pregnancies of and/or births by the database cow; and
the database allows the determination of whether a cow is open based at least in part on historical flow cytometry data and new flow cytometry data.

30. The method of claim 29, further comprising:
providing a second biological sample comprising leukocytes of a second cow, wherein the leukocytes of the second cow are characterized by the fact that they were drawn from the second cow after the second cow underwent an initial implementation of a method to establish pregnancy;
fixing and permeabilizing the leukocytes of the second cow;
contacting the leukocytes of the second cow the binding agent, thereby producing a second fluorescently-labeled sample;
analyzing the second fluorescently-labeled sample by flow cytometry, thereby obtaining new flow cytometry data for the second cow;
determining whether the second cow is open based on the database and the new flow cytometry data for the second cow, thereby obtaining a determination that the second cow is open or not open; and
implementing a subsequent method to establish pregnancy in the second cow within 5 days of obtaining the determination if the determination is that the second cow is open, wherein:
if the database were not updated with the new flow cytometry data for the first cow, then (a) the determination would not have been that the second cow was open, and (b) the subsequent method to establish pregnancy in the second cow would not have been implemented within 5 days of the determination.

31. The method of claim 30, further comprising implementing a software algorithm after updating the database with the new flow cytometry data for the first cow, wherein:
the software algorithm is configured to minimize the probability of either a false positive determination or a false negative determination; and either the software algorithm is implemented as part of the determining step to obtain a determination that is specific to the second cow; or
the software algorithm is implemented prior to the determining step; implementation of the software algorithm sets generally-applicable threshold values; and the determining step comprises comparing the new flow cytometry data for the second cow and the database entry for the second cow with the generally-applicable threshold values.

32. The method of claim 31, wherein the database information of each database entry further comprises one or more of: (7) date(s) on which the database cow of the database entry was administered antibiotics, if any, (9) dates on which the database cow was administered vaccines, if any, (10) dates on which the database cow was administered a pharmaceutical agent other than antibiotics or vaccines, if any, (11) medical history of the database cow related to parasite infection, viral infection, bacterial infection, and/or trauma (12) body temperature measurements of the database cow, (13) diet of the database cow, (14) genetic information of the database cow, and (15) climate and/or weather information associated with the geographic location.

33. A method for improved reproductive management using a remote tracking and computer environment, comprising:
providing a sample collection container having a first unique identifier, wherein the first unique identifier is visually readable (e.g., an alphanumeric) or electronically readable (e.g., a barcode such as a QR code);
inserting an electronically-identifiable sample from an electronically-identifiable cow into the sample collection container, wherein: (a) the electronically-identifiable cow is associated with a second unique identifier; (b) the second unique identifier is visually readable (e.g., an ear tag) or electronically readable (e.g., a RFID tag); (c) the electronically-identifiable cow is the first cow or the second cow, and the electronically-identifiable sample is the sample comprising leukocytes of the first cow or the second cow, respectively; and (d) the electronically-identifiable sample is electronically-identifiable because it is inserted into the sample collection container;
electronically recording the first unique identifier and the second unique identifier onto mobile storage media of a mobile device, thereby generating updated mobile storage media;
wirelessly transmitting the first unique identifier and the second unique identifier from the updated mobile storage media to fixed storage media of a testing site computer, thereby generating updated fixed storage media;
assigning the electronically-identifiable sample to an assigned sample well at the testing site;
electronically recording the assigned sample well onto the updated fixed storage media, thereby generating assigned fixed storage media;
transferring the electronically-identifiable sample from the sample collection container to the assigned sample well; and
performing the method of claim 29 at the testing site.

34. The method of claim 33, wherein wirelessly transmitting the first unique identifier and the second unique identifier from the updated mobile storage media to the fixed storage media comprises:
wirelessly transmitting the first unique identifier and the second unique identifier from the updated mobile storage media to remote storage media of a remote server, thereby generating updated remote storage media; and
wirelessly transmitting the first unique identifier and the second unique identifier from the updated remote storage media to the fixed storage media, thereby generating the updated fixed storage media.
